(19) United States
(12) Patent Application Publication
(10) Pub. No.: US 2024/0294471 A1
(43) Pub. Date: Sep. 5, 2024

(54) ROS-RESPONSIVE CAPTOPRIL-CINNAMALDEHYDE PRODRUGS AND COMPOSITIONS AND METHODS THEREOF

(71) Applicant: The University of Hong Kong, Hong Kong (CN)

(72) Inventors: Weiping Wang, Hong Kong (CN); Yaming Zhang, Hong Kong (CN)

(21) Appl. No.: 18/569,361

(22) PCT Filed: Jul. 6, 2022
(86) PCT No.: PCT/CN2022/104160

§ 371 Date: Dec. 12, 2023

Related U.S. Application Data
(60) Provisional application No. 63/221,318, filed on Jul. 13, 2021.

(51) Int. Cl. C07D 207/16; A61K 31/4025; A61K 45/06; A61P 29/00

(52) U.S. Cl. CPC C07D 207/16; A61K 31/4025; A61K 45/06; A61P 29/00

(57) ABSTRACT

ROS-responsive prodrugs and methods of making thereof are disclosed. These prodrugs can contain two captopril moieties and one cinnamaldehyde moiety that are linked by a thioacetal linking moiety; upon cleavage of the thioacetal linking moiety by ROS, the prodrugs can release two captopril or captopril derivatives and one cinnamaldehyde or cinnamaldehyde derivative. Pharmaceutical formulations in a unit dosage form suitable for the delivery of the prodrugs to a subject in need thereof are disclosed. The pharmaceutical formulations may include one or more additional anti-inflammatory active agents in their free form. The pharmaceutical formulation can be administered by oral administration, parenteral administration, inhalation, mucosal administration, or a combination thereof. Methods for preventing or treating an inflammatory disease or disorder, or treating or ameliorating one or more symptoms associated with an inflammatory disease or disorder in a subject are also disclosed. The inflammatory disease or disorder can be rheumatoid arthritis.

### FIELD OF THE INVENTION

This invention is generally in the field of synthetic prodrugs and their use for treating anti-inflammatory diseases or disorders.

### BACKGROUND OF THE INVENTION

Rheumatoid arthritis (“RA”) is a common autoimmune disease that is characterized by chronic inflammation in the synovial membrane of joints. Disease progress gradually causes joint destruction and disability, resulting in a poor quality of patients' life. Current available drugs for the treatment of RA are incapable of recovering the damaged joints to the original state, and usually cause local and systemic adverse effects due to the instability and off-target toxicity of these drugs.

Prodrugs are inactive compounds that undergo a metabolic conversion to release the pharmacologically active agents. Prodrugs can be used to improve undesirable absorption, distribution, metabolism, excretion, toxicity (ADMET) properties. Elevated levels of reactive oxygen species (“ROS”) are associated with inflammation, such as in patients with RA, which can be used as a stimulus for prodrug activation. However, there are no ROS-responsive prodrugs available to treat RA in the market.

Some studies have reported ROS-responsive prodrugs for RA treatment. However, the synthetic routes of these prodrugs are complicated and undesirable byproducts are generated after prodrug activation.

There remains a need to develop prodrugs that can release anti-inflammatory agents upon ROS stimulation.

Therefore, it is the object of the present invention to provide ROS-responsive prodrugs.

It is a further object of the present invention to provide methods of making the ROS-responsive prodrugs.

It is a further object of the present invention to provide methods of using the ROS-responsive prodrugs.

### SUMMARY OF THE INVENTION

Synthetic compounds as prodrugs and their methods of making and using are described.

Generally, the prodrugs contain two or more active agent moieties linked by a ROS-responsive thioacetal linking moiety. Upon activation by ROS, the thioacetal linking moiety of the prodrug is cleaved and thereby releases two or more active agents in their free from. In some forms, the two or more active agents in their free form can have anti-inflammatory properties and are suitable for use in the prevention and/or treatment of a variety of anti-inflammatory diseases or disorders, and/or amelioration of the symptom(s) of a variety of anti-inflammatory diseases or disorders, such as rheumatoid arthritis. In some forms, the prodrugs contain two captopril moieties and one cinnamaldehyde moiety that are linked by a thioacetal linking moiety; upon cleavage by ROS, the prodrugs can release two captopril or captopril derivatives and one cinnamaldehyde or cinnamaldehyde derivative.

In some forms, the compound can have the structure of Formula I″a:

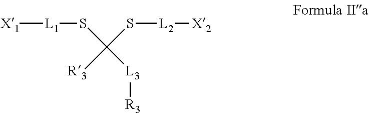

wherein: (a) L₁, L₂, and L₃ can be independently a bond (single, double, or triple), a substituted or unsubstituted alkyl, a substituted or unsubstituted alkenyl, an amino, an amido, a carbonyl, an alkoxyl, a polyether, a thioether; (b) X′₁ and X′₂ can be independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl); (c) R₃ can be a substituted or unsubstituted aryl, a substituted or unsubstituted polyaryl, a substituted or unsubstituted heteroaryl, or a substituted or unsubstituted polyheteroaryl; (d) R′₃ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and (e) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

In some forms of Formula I″a, X′₁-L₁- and X′₂-L₂- can be independently a moiety of Formula I″b:

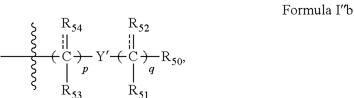

where: (a) Y′ can be a bond (single, double, or triple) or NR₅₅, and R₅₅ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (b) each occurrence of R₅₁ and R₅₃ can be independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (c) each occurrence of R₅₂ and R₅₄ can be independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (d) R₅₀ can be hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl); (e) p and q can be independently an integer from 0 to 10, from 0 to 8, from 0 to 6, from 0 to 4, from 0 to 3, from 0 to 2, or 0 or 1; and (f) the substituents can be as defined above for Formula I″a.

In some forms, the moiety of Formula I″b can have the structure of:

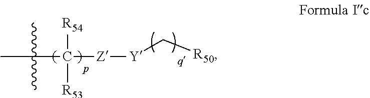

where: (a) each occurrence of R₅₃ and R₅₄ can be independently hydrogen, a substituted or unsubstituted alkyl, a substituted or unsubstituted aryl, a substituted or unsubstituted alkylaryl, a substituted or unsubstituted heterocyclyl, an amino, or a carbonyl; (b) Z′ can be a single bond or

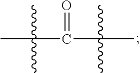 [SMILES: *C(*)=O]

(c) Y′ can be a single bond or NR₅₅, and R₅₅ is hydrogen or a substituted or unsubstituted alkyl; (d) R₅₀ can be a carboxyl or a substituted or unsubstituted heterocyclyl (monoheterocyclyl or polyheterocyclyl); (e) p′ and q′ can be independently an integer from 0 to 3; and (f) the substituents can be as defined above for Formula I″a.

In some forms, the prodrug can have the structure of Formula I:

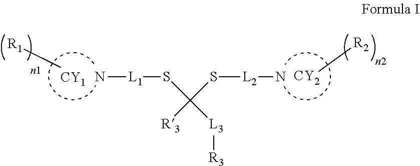

where: (a) L₁, L₂, and L₃ can be independently a bond (single, double, or triple), a substituted or unsubstituted alkyl, a substituted or unsubstituted alkenyl, an amino, an amido, a carbonyl, an alkoxyl, a polyether, a thioether; (b) CY₁ and CY₂ can be independently a substituted or unsubstituted heteroaryl, a substituted or unsubstituted polyheteroaryl, or a substituted or unsubstituted heterocyclyl; (c) R₁ and R₂ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether; (d) n1 and n2 can be independently an integer from 0 to 10; (e) R₃ can be a substituted or unsubstituted aryl, a substituted or unsubstituted polyaryl, a substituted or unsubstituted heteroaryl, or a substituted or unsubstituted polyheteroaryl; (f) R′₃ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and (g) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol. In Formula I, (R₁)ₙ₁—CY₁N-L₁-, (R₂)ₙ₂—CY₂N-L₂-, and R₃-L₃- constitute the active agent moieties and

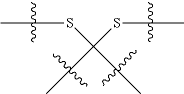 [SMILES: *SC(*)(*)S*]

constitutes the ROS-responsive thioacetal linking moiety.

In some forms, the prodrug can have the structure of Formula II:

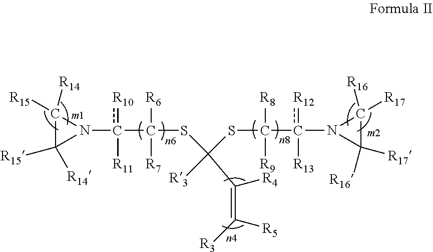

where: (a) each occurrence of R₄-R₉, R₁₄-R₁₇, and R₁₄′-R₁₇′ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (b) m1 and m2 can be independently an integer from 1 to 9; (c) n4, n6, and n8 can be independently an integer from 1 to 10; (d) R₁₀ and R₁₂ can be independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (e) R₁₁ and R₁₃ can be independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (f) R′₃, R₃, and the substituents can be as defined above for Formula I. In Formula II,

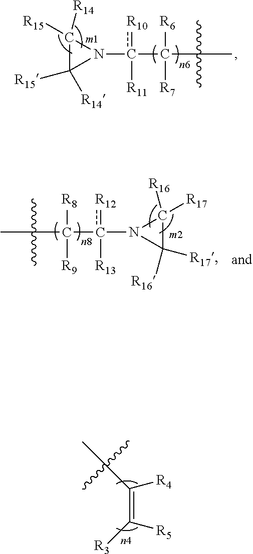

constitute the active agent moieties and

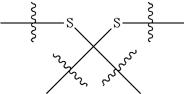 [SMILES: *SC(*)(*)S*]

constitutes the ROS-responsive thioacetal linking moiety.

In some forms, the prodrug can have the structure of Formula III:

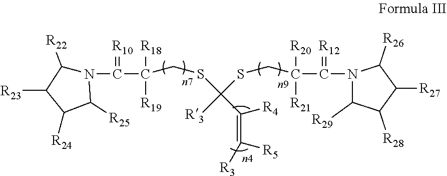

where: (a) n7 and n9 can be independently an integer from 0 to 8; (b) R₁₈, R₁₉, R₂₀, and R₂₁ can be independently hydrogen, a substituted or unsubstituted alkyl, a substituted or unsubstituted aryl, an alkoxyl, a hydroxyl, or a halogen; R₁₀ and R₁₂ can be independently oxygen or sulfur; R₂₂-R₂₉ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol; (e) n4 can be an integer from 1 to 4; (f) R′₃, R₃-R₅, and the substituents can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol. In Formula III,

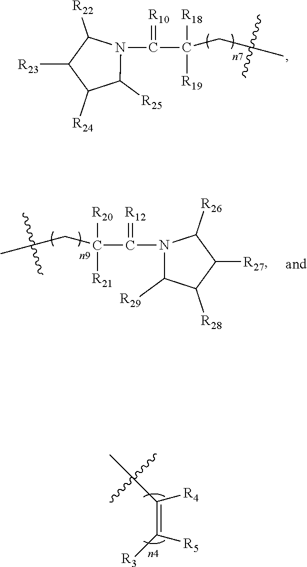

constitute the active agent moieties and

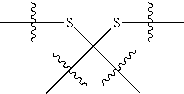 [SMILES: *SC(*)(*)S*]

constitutes the ROS-responsive thioacetal linking moiety.

In some forms of Formula III, at least one of R₁₈ and R₁₉ and/or at least one of R₂₀ and R₂₁ can be a substituted or unsubstituted alkyl, a substituted or unsubstituted aryl, an alkoxyl, a hydroxyl, or a halogen. In some forms of Formula III, at least one of R₁₈ and R₁₉ and/or at least one of R₂₀ and R₂₁ can be a substituted or unsubstituted alkyl. In some forms of Formula III, at least one of R₂₂ and R₂₅ and/or at least one of R₂₆ and R₂₈ can be

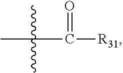

and R₃₁ is a hydroxyl, an alkoxyl, or an amino.

In some forms of Formulae I″a, I″b, I, II, and/or III, R′₃ can be hydrogen. In some forms of Formulae II and/or III, R₄ and R₅ can be independently hydrogen, a substituted or unsubstituted alkyl, a substituted or unsubstituted aryl, an alkoxyl, a hydroxyl, or a halogen.

In some forms of Formulae I″a, I″b, I, II, and/or III, R₃ can be

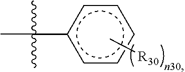

n30 can be an integer from 0 to 5, and R₃₀ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether.

In some forms, the prodrug can have the structure of:

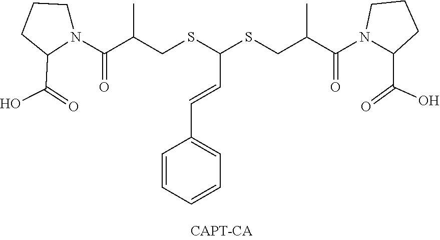 [SMILES: CC(CSC(/C=C/c1ccccc1)SCC(C)C(=O)N1CCCC1C(=O)O)C(=O)N1CCCC1C(=O)O]

Pharmaceutical compositions and pharmaceutical formulations in a unit dosage form suitable for the delivery of the prodrugs and their preparation are disclosed. Generally, the pharmaceutical composition or formulation contains one or more of the prodrugs and a suitable pharmaceutically acceptable carrier and/or excipient. The prodrugs in the pharmaceutical compositions or formulations are in an effective amount for preventing or treating an inflammatory disease or disorder, or treating or ameliorating one or more symptoms associated with an inflammatory disease or disorder in a subject in need thereof. In some forms, the pharmaceutical composition or formulation contains polymeric particles or micelles as carriers, where the one or more prodrugs are encapsulated in, conjugated to, and/or complexed with the polymeric particles or micelles. In some forms, the pharmaceutical composition or formulation can further contain one or more additional active agents, such as one or more additional anti-inflammatory agents.

The methods of making the prodrugs disclosed herein can include (i) cooling a reaction mixture at a suitable temperature for a period of time sufficient to form a product containing the prodrug. The reaction mixture can contain two or more reactants and a solvent. In some forms, the reaction mixture contains two captopril or captopril derivatives and one cinnamaldehyde or cinnamaldehyde derivative. The two captopril or captopril derivatives can be the same or different. In some forms, the reaction mixture can further contain a catalyst, such as trifluoroacetic acid, p-toluene sulfonic acid, or boron trifluoride, or a combination thereof.

In some forms, the method further includes mixing the two or more reactants, optionally the catalyst, and the solvent to form the reaction mixture prior to step (i) and/or purifying the product containing the prodrug subsequent to step (i).

The methods of using the prodrugs for preventing or treating an inflammatory disease or disorder, or treating or ameliorating one or more symptoms associated with an inflammatory disease or disorder in a subject in need thereof can include administering to the subject the pharmaceutical formulation containing one or more of the disclosed prodrugs. The administration step can occur one or more times. The subject is typically a mammal, such as a human. In some forms of the method, the pharmaceutical formulation can be administered by oral administration, parenteral administration, inhalation, mucosal administration, topical or a combination thereof.

In some forms of the method, following a single administration or more than one administration of the pharmaceutical formulation to the subject, an effective amount of the products to prevent or treat the inflammatory disease or disorder, or treat or ameliorate one or more symptoms associated with the inflammatory disease or disorder in the subject is administered to the subject, as shown by one or more known clinical and/or biochemical measurements, such as Ritchie articular index (AI), early-morning stiffness, clinical score, grip strength, pain score, supine blood pressure, hemoglobin (Hb), white blood cell count, platelets, immunoglobulin M, plasma viscosity, C-reactive protein, or histidine, or a combination thereof.

In some forms, the method further includes administering to the subject one or more additional active agents prior to, during, and/or subsequent to step (i). The one or more additional active agents can be one or more anti-inflammatory agents.

Methods for treating macrophages in a subject in need thereof are also disclosed. The method can include (i) administering to the subject the pharmaceutical formulation containing one or more of the disclosed prodrugs, where step (i) occurs one or more times.

In some forms of the method, following a single administration or more than one administration of the pharmaceutical formulation to the subject, an effective amount of the prodrugs to reduce production of nitric oxide by macrophages and/or reduce release of one or more proinflammatory cytokines from the macrophages is administered to the subject, compared with the production of nitric oxide by macrophages and/or release of the one or more proinflammatory cytokines from the macrophage in a control not treated with the prodrugs, tested under the same condition.

Methods for treating or ameliorating one or more symptoms associated with a cancer and/or hypertension in a subject in need thereof are also disclosed.

### BRIEF DESCRIPTION OF THE DRAWINGS

FIG. 1 is a bar graph showing the cytotoxicity of CAPT-CA against Raw264.7 cells. The cells were incubated with the indicated concentration of prodrug for 24 h with or without LPS stimulation. Data were presented as means±SD (n=3).

FIG. 2 is a bar graph showing the relative nitric oxide generation in the supernatant from Raw 264.7 cells. The cells were incubated with the indicated concentration of prodrug for 24 h with or without LPS stimulation. Data were presented as means±SD (n=3).

FIGS. 3A-3C are bar graphs showing the quantification of TNF-α (FIG. 3A), IL-1β (FIG. 3B), and IL-6 (FIG. 3C) protein levels in the supernatant from Raw 264.7 cells. The cells were incubated with the indicated concentration of prodrug for 24 h with or without LPS stimulation. Data were presented as means±SD (n=3).

FIGS. 4A-4C are bar graphs showing the quantification of TNF-α (FIG. 4A), IL-6 (FIG. 4B), and IL-1β (FIG. 4C) mRNA levels from Raw 264.7 cells. The cells were incubated with the indicated concentration of prodrug for 24 h with or without LPS stimulation. Data were presented as means±SD (n=3).

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with the permitted valence of the substituted atom and the substituent and that the substitution results in a stable compound, i.e. a compound that does not spontaneously undergo transformation such as by rearrangement, cyclization, elimination, etc.

“Alkyl,” as used herein, refers to the radical of saturated aliphatic groups, including straight-chain alkyl groups, branched chain alkyl, and cycloalkyl (alicyclic). In some forms, a straight-chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

The term “alkyl” as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof. The term “alkyl” also includes “heteroalkyl”.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. “Heteroalkyl,” as used herein, refers to straight or branched chain, or cyclic carbon-containing alkyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulfur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl”.

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C═C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl”.

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. “Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

The term “aryl” as used herein is any C₅-C₂₆ carbon-based aromatic group, heteroaromatic, fused aromatic, or fused heteroaromatic. For example, “aryl,” as used herein can include 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused aromatic rings”), wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF3, —CH₂—CF₃, —CCl₃), —CN, aryl, heteroaryl, and combinations thereof.

“Heterocycle” and “heterocyclyl” are used interchangeably and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₃₀-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl”.

The term “substituted heteroaryl” refers to a heteroaryl group in which one or more hydrogen atoms on one or more heteroaromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF3, —CH₂—CF₃, —CCl₃), —CN, aryl, heteroaryl, and combinations thereof.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof. For example, a “polyaryl” can be polycyclic ring systems having two or more cyclic rings in which two or more carbons are common to two adjoining rings (i.e., “fused aromatic rings”), wherein two or more of the rings are aromatic. When two or more heteroaryls are involved, the chemical moiety can be referred to as a “polyheteroaryl.”

The term “substituted polyaryl” refers to a polyaryl in which one or more of the aryls, heteroaryls are substituted, with one or more substituents. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. When two or more heteroaryls are involved, the chemical moiety can be referred to as a “substituted polyheteroaryl.”

The term “cyclic ring” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, a substituted or unsubstituted heterocyclyl, a substituted or unsubstituted aryl, a substituted or unsubstituted polyaryl, a substituted or unsubstituted heteroaryl, and a substituted or unsubstituted polyheteroaryl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, heterocyclyls, aryls, heteroaryl, polyaryls, and polyheteroaryls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by oxygen as defined below. Accordingly, the substituent of an alkyl that renders that alkyl an ether is or resembles an alkoxyl, such as can be represented by one of —O-alkyl, —O-alkenyl, —O-alkynyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

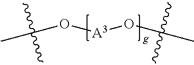

where A³, A², and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art-recognized and refers to a compound of the formula —OR^{v} wherein R^{v} is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

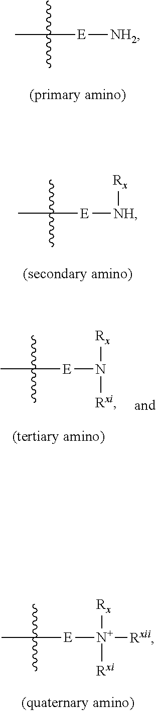

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, Rₓᵢ, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{X}, R^{xi}, and R^{xii} with the N⁺ to which they are attached complete a heterocyclyl or heteroaryl having from 3 to 14 atoms in the ring structure.

The terms “amide” or “amido” are used interchangeably, refer to both “unsubstituted amido” and “substituted amido” and are represented by the general formula:

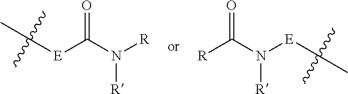

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed.

“Carbonyl,” as used herein, is art-recognized and includes such moieties as can be represented by the general formula:

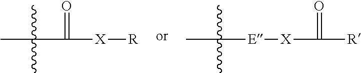

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Where X is oxygen and R is defines as above, the moiety is also referred to as a carboxyl group. When X is oxygen and R is hydrogen, the formula represents a “carboxylic acid”. Where X is oxygen and R′ is hydrogen, the formula represents a “formate”. Where X is oxygen and R or R′ is not hydrogen, the formula represents an “ester”. In general, where the oxygen atom of the above formula is replaced by a sulfur atom, the formula represents a “thiocarbonyl” group. Where X is sulfur and R or R′ is not hydrogen, the formula represents a “thioester”. Where X is sulfur and R is hydrogen, the formula represents a “thiocarboxylic acid”. Where X is sulfur and R′ is hydrogen, the formula represents a “thioformate”. Where X is a bond and R is not hydrogen, the above formula represents a “ketone”. Where X is a bond and R is hydrogen, the above formula represents an “aldehyde”.

The term “substituted carbonyl” refers to a carbonyl, as defined above, wherein one or more hydrogen atoms in R, R′ or a group to which the moiety

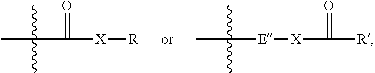

is attached, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “carboxyl” is as defined above for carbonyl and is defined more specifically by the formula —R^{iv}COOH, wherein R^{iv} is a substituted or unsubstituted alkyl, a substituted or unsubstituted alkenyl, a substituted or unsubstituted alkynyl, a substituted or unsubstituted heterocyclyl, a substituted or unsubstituted alkylaryl, a substituted or unsubstituted arylalkyl, a substituted or unsubstituted aryl, a substituted or unsubstituted polyaryl, a substituted or unsubstituted polyheteroaryl, or a substituted or unsubstituted heteroaryl.

The term “substituted carboxyl” refers to a carboxyl, as defined above, wherein one or more hydrogen atoms in R^{iv} are substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphanyl” is represented by the formula

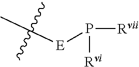

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonium” is represented by the formula

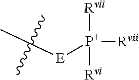

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “phosphonyl” is represented by the formula

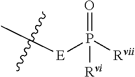

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “substituted phosphonyl” represents a phosphonyl in which E, R^{vi} and R^{vii} are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfinyl” is represented by the formula

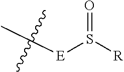

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “sulfonyl” is represented by the formula

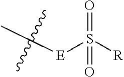

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “substituted sulfonyl” represents a sulfonyl in which E, R, or both, are independently substituted. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

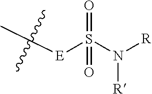

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8.

The term “silyl group” as used herein is represented by the formula —SiRR′R″, where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. “Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “hydroxyl” and “hydroxy” are used interchangeably and are represented by —OH.

The term “oxo” refers to ═O bonded to a carbon atom.

The terms “cyano” and “nitrile” are used interchangeably to refer to —CN.

The term “nitro” refers to —NO₂.

The term “phosphate” refers to —O—PO₃.

The term “azide” or “azido” are used interchangeably to refer to —N₃.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within the second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

### II. Compositions

Synthetic compounds that can be used as prodrugs have been developed. These compounds (also referred to herein as “prodrugs”) can release two or more active agents upon stimulation by reactive oxygen species (“ROS”), such as H₂O₂, HO·, and O₂⁻. The released active agents have anti-inflammatory properties and should be suitable for use in the prevention and/or treatment of a variety of anti-inflammatory diseases or disorders, and/or amelioration of the symptom(s) of a variety of anti-inflammatory diseases or disorders. In particular, these compounds are ROS-responsive prodrugs that can be cleaved by ROS at inflammatory sites and release two or more anti-inflammatory active agents and are suitable for use in the prevention and/or treatment of rheumatoid arthritis (“RA”) and/or amelioration of the symptom(s) of RA.

The prodrugs can be formed by reaction between active agent(s) containing sulfhydryl groups and active agent(s) containing aldehyde or ketone groups. The sulfhydryl groups and aldehyde or ketone groups can react to form a ROS-responsive linking moiety, i.e. thioacetal linking moiety. Accordingly, the formed prodrugs contain two or more active agent moieties that correspond to the unreacted portions of the active agents; the active agent moieties are linked by the ROS-responsive linking moiety. Due to the abundant ROS generated at inflammatory sites, the thioacetal linking moiety can be cleaved by the ROS, thereby release the two or more active agents in free form, without any byproduct generation. These prodrugs can increase the stability and bioavailability of the active agents and reduce the side effects of these active agents, compared with the same active agents administered in their free form.

Pharmaceutical compositions and formulations containing the prodrugs are also disclosed.

### A. Prodrugs

### 1. Prodrug Structures

The prodrugs can have the structures of Formula I′:

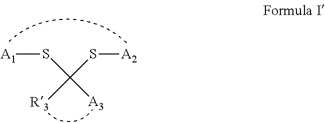

where each of A₁, A₂, and A₃ can be an active agent moiety; R′₃ can be absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; [symbol] can be independently absent or a bond (single, double, or triple); and the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol; and the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

In some forms, the prodrugs can have the structure of Formula I″:

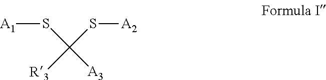

where A₁, A₂, A₃, and R′₃ can be as defined above for Formula I′.

In some forms of Formula I″, A₁ and A₂ can be independently a moiety of penicillamine or a penicillamine derivative, a moiety of tiopronin or a tiopronin derivative, a moiety of methimazole or a methimazole derivative, a moiety of omapatrilat or an omapatrilat derivative, or a moiety of captopril or a captopril derivative, and A₃ can be a moiety of cinnamaldehyde or a cinnamaldehyde derivative.

In some forms, the prodrugs can have the structure of Formula I″a:

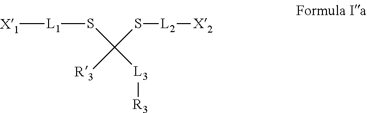

where: (a) L₁, L₂, and L₃ can be independently absent, a bond (single, double, or triple), a substituted or unsubstituted alkyl, a substituted or unsubstituted alkenyl, an amino, an amido, a carbonyl, an alkoxyl, a polyether, a thioether; (b) X′₁ and X′₂ can be independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl); (c) R₃ can be a substituted or unsubstituted aryl, a substituted or unsubstituted polyaryl, a substituted or unsubstituted heteroaryl, or a substituted or unsubstituted polyheteroaryl; (d) R′₃ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and (e) the substituents can be as defined above for Formula I′. In Formula I″a, X′₁-L₁-, X′₂-L₂-, and R₃-L₃- constitute the active agent moieties and

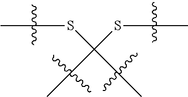 [SMILES: *SC(*)(*)S*]

constitutes the ROS-responsive thioacetal linking moiety.

In some forms of Formula I″a, X′₁-L₁- and X′₂-L₂- can be independently a moiety of Formula I″b:

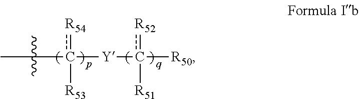

where: (a) Y′ can be a bond (single, double, or triple) or NR₅₅, and R₅₅ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (b) each occurrence of R₅₁ and R₅₃ can be independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (c) each occurrence of R₅₂ and R₅₄ can be independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (d) R₅₀ can be hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl); (e) p and q can be independently an integer from 0 to 10, from 0 to 8, from 0 to 6, from 0 to 4, from 0 to 3, from 0 to 2, or 0 or 1; and (f) the substituents can be as defined above for Formula I′.

In some forms of Formula I″b, (a) Y′ can be a bond (single, double, or triple) or NR₅₅, and R₅₅ can be hydrogen or a substituted or unsubstituted alkyl, a substituted or unsubstituted aryl, a substituted or unsubstituted heterocyclyl, an amino, an alkoxy, or a carbonyl; (b) each occurrence of R₅₁ and R₅₃ can be independently absent, hydrogen, a substituted or unsubstituted alkyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an amido, an amino, an alkoxy, or a carbonyl; and (c) each occurrence of R₅₂ and R₅₄ can be independently oxygen, hydrogen, a substituted or unsubstituted alkyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an amido, an amino, an alkoxy, or a carbonyl.

In some forms of Formula I″b, (a) Y′ can be a bond (single, double, or triple) or NR₅₅, and R₅₅ can be hydrogen or a substituted or unsubstituted alkyl; (b) each occurrence of R₅₁ and R₅₃ can be independently absent, hydrogen, a substituted or unsubstituted alkyl, a substituted or unsubstituted aryl, a substituted or unsubstituted heterocyclyl, an amido, an amino, or a carbonyl; (c) each occurrence of R₅₂ and R₅₄ can be independently oxygen, hydrogen, a substituted or unsubstituted alkyl, a substituted or unsubstituted aryl, a substituted or unsubstituted heterocyclyl, an amido, an amino, or a carbonyl; (d) p and q can be independently an integer from 0 to 4, from 0 to 3, from 0 to 2, or 0 or 1; and (e) R₅₀ can be hydrogen, a carboxyl, or a substituted or unsubstituted heterocyclyl (monoheterocyclyl or polyheterocyclyl).

In some forms of Formula I″b, the moiety can have the structure of Formula I″c:

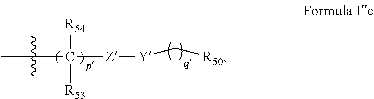

where: (a) each occurrence of R₅₃ and R₅₄ can be independently hydrogen, a substituted or unsubstituted alkyl, a substituted or unsubstituted aryl, a substituted or unsubstituted alkylaryl, a substituted or unsubstituted heterocyclyl, an amino, or a carbonyl; (b) Z′ can be a single bond or

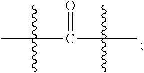 [SMILES: *C(*)=O]

(c) Y′ can be a single bond or NR₅₅, and R₅₅ can be hydrogen or a substituted or unsubstituted alkyl; (d) R₅₀ can be a carboxyl or a substituted or unsubstituted heterocyclyl (monoheterocyclyl or polyheterocyclyl); and (e) p′ and q′ can be independently an integer from 0 to 3.

In some forms of Formula I″a, X′₁-L₁- and X′₂-L₂- can be independently a moiety of penicillamine, a moiety of tiopronin, a moiety of methimazole, a moiety of omapatrilat, or a moiety of captopril or a captopril derivative:

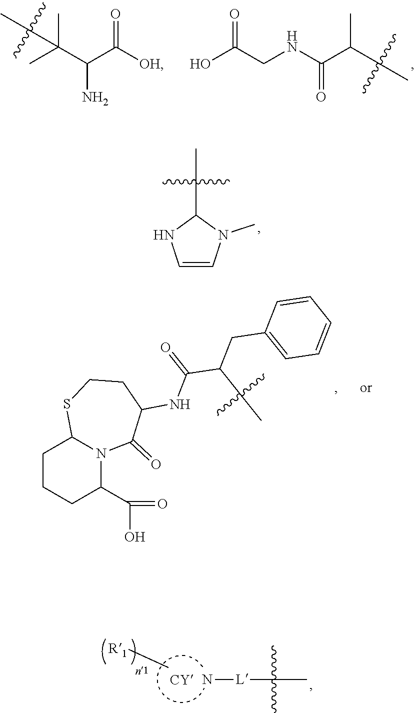

where L′ can be independently a bond (single, double, or triple), a substituted or unsubstituted alkyl, a substituted or unsubstituted alkenyl, an amino, an amido, a carbonyl, an alkoxyl, a polyether, a thioether; (b) CY′ can be independently a substituted or unsubstituted heteroaryl, a substituted or unsubstituted polyheteroaryl, or a substituted or unsubstituted heterocyclyl; (c) R′₁ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether; (d) n′1 can be independently an integer from 0 to 10; and (e) the substituents can be as defined above for Formula I′.

In some forms of Formula I″, A₁ and A₂ can be independently a moiety of captopril or a captopril derivative and A₃ can be a moiety of cinnamaldehyde or a cinnamaldehyde derivative. In these forms, the prodrugs can have the structure of Formula I:

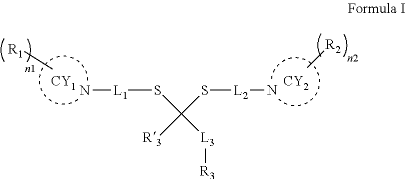

where: (a) L₁, L₂, and L₃ can be independently a bond (single, double, or triple), a substituted or unsubstituted alkyl, a substituted or unsubstituted alkenyl, an amino, an amido, a carbonyl, an alkoxyl, a polyether, a thioether; (b) CY₁ and CY₂ can be independently a substituted or unsubstituted heteroaryl, a substituted or unsubstituted polyheteroaryl, or a substituted or unsubstituted heterocyclyl; (c) R₁ and R₂ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether; (d) n1 and n2 can be independently an integer from 0 to 10; (e) R₃ can be a substituted or unsubstituted aryl, a substituted or unsubstituted polyaryl, a substituted or unsubstituted heteroaryl, or a substituted or unsubstituted polyheteroaryl; (f) R′₃ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and (g) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol. In Formula I, (R₁)ₙ₁—CY₁N-L₁-, (R₂)ₙ₂—CY₂N-L₂-, and R₃-L₃- constitute the active agent moieties and

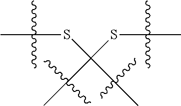 [SMILES: *SC(*)(*)S*]

constitutes the ROS-responsive thioacetal linking moiety.

In some forms, the prodrugs can have the structure of Formula II:

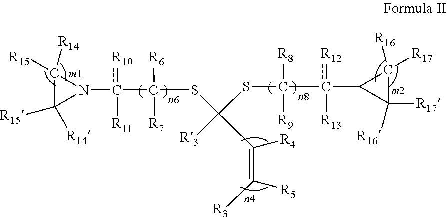

where: (a) each occurrence of R₄-R₉, R₁₄-R₁₇, and R₁₄′-R₁₇′ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (b) m1 and m2 can be independently an integer from 1 to 9; (c) n4, n6, and n8 can be independently an integer from 1 to 10; (d) R₁₀ and Ru can be independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (e) R₁₁ and R₁₃ can be independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (f) R′₃, R₃, and the substituents can be as defined above for Formula I. In Formula II,

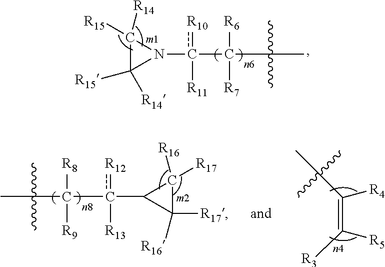

constitute the active agent moieties and

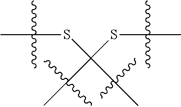 [SMILES: *SC(*)(*)S*]

constitutes the ROS-responsive thioacetal linking moiety.

In some forms, the prodrugs can have the structure of Formula III:

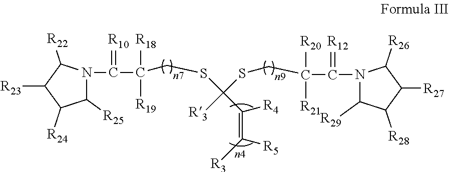

where: (a) n7 and n9 can be independently an integer from 0 to 8; R₁₈, R₁₉, R₂₀, and R₂₁ can be independently hydrogen, a substituted or unsubstituted alkyl, a substituted or unsubstituted aryl, an alkoxyl, a hydroxyl, or a halogen; (c) R₁₀ and R₁₂ can be independently oxygen or sulfur; (d) R₂₂-R₂₉ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol; (e) n4 can be an integer from 1 to 8, 1 to 6, 1 to 4, 1 to 3, or 1 or 2; (f) R′₃, R₃-R₅, and the substituents can be as defined above for Formula II. In Formula III,

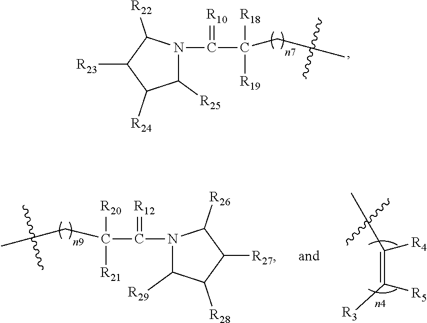

constitute the active agent moieties and

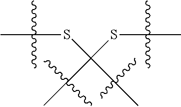 [SMILES: *SC(*)(*)S*]

constitutes the ROS-responsive thioacetal linking moiety.

In some forms of Formula III, at least one of R₁₈ and R₁₉ and/or at least one of R₂₀ and R₂₁ can be a substituted or unsubstituted alkyl, a substituted or unsubstituted aryl, an alkoxyl, a hydroxyl, or a halogen. In some forms of Formula III, at least one of R₁₈ and R₁₉ and/or at least one of R₂₀ and R₂₁ can be a substituted or unsubstituted alkyl.

In some forms of Formula III, at least one of R₂₂ and R₂₅ and/or at least one of R₂₆ and R₂₈ can be

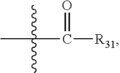

and R₃₁ can be a hydroxyl, an alkoxyl, or an amino.

In some forms of Formulae II and/or III, R₄ and R₅ can be independently hydrogen, a substituted or unsubstituted alkyl, a substituted or unsubstituted aryl, an alkoxyl, a hydroxyl, or a halogen. In some forms of Formulae II and/or III, R₄ and R₅ can be independently hydrogen or a substituted or unsubstituted alkyl.

In some forms of Formulae I′, I″, I″a, I, II, and/or III, R′₃ can be hydrogen. In some forms of Formulae I″a, I, II, and/or III, R₃ can be

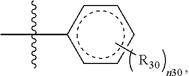

n30 can be an integer from 0 to 5, from 0 to 4, from 0 to 3, from 0 to 2, or 0 or 1, and R₃₀ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether. In some forms of Formulae I″a, I, II, and/or III, R₃ can be

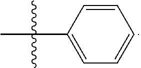 [SMILES: *c1ccccc1]

For any of Formulae I′, I″, I″a, I″b, I″c, and I-III, the alkyl can be a linear alkyl, a branched alkyl, or a cyclic alkyl (either monocyclic or polycyclic). Exemplary alkyl includes a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, C₁-C₂ alkyl group, a branched C₃-C₉, C₃-C₉, C₃-C₈, C₃-C₇, C₃-C₆, C₃-C₅, C₃-C₄ alkyl group, or a cyclic C₃-C₉, C₃-C₉, C₃-C₈, C₃-C₇, C₃-C₆, C₃-C₅, C₃-C₄ alkyl group.

For any of Formulae I′, I″, I″a, I″b, I″c, and I-III, the alkenyl can be a linear alkenyl, a branched alkenyl, or a cyclic alkenyl (either monocyclic or polycyclic).

Exemplary alkenyl includes a linear C₁-C₃₀ alkenyl, a branched C₄-C₃₀ alkenyl, a cyclic C₃-C₃₀ alkenyl, a linear C₁-C₂₀ alkenyl, a branched C₄-C₂₀ alkenyl, a cyclic C₃-C₂₀ alkenyl, a linear C₁-C₁₀ alkenyl, a branched C₄-C₁₀ alkenyl, a cyclic C₃-C₁₀ alkenyl, a linear C₁-C₆ alkenyl, a branched C₄-C₆ alkenyl, a cyclic C₃-C₆ alkenyl, a linear C₁-C₄ alkenyl, cyclic C₃-C₄ alkenyl, such as a linear C₁-C₁₀, C₁-C₉, C₁-C₈, C₁-C₇, C₁-C₆, C₁-C₅, C₁-C₄, C₁-C₃, C₁-C₂ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group.

For any of Formulae I′, I″, I″a, I″b, I″c, and I-III, the alkynyl can be a linear alkynyl, a branched alkynyl, or a cyclic alkynyl (either monocyclic or polycyclic). Exemplary alkynyl includes a linear C₁-C₃₀ alkynyl, a branched C₄-C₃₀ alkynyl, a cyclic C₃-C₃₀ alkynyl, a linear C₁-C₂₀ alkynyl, a branched C₄-C₂₀ alkynyl, a cyclic C₃-C₂₀ alkynyl, a linear C₁-C₁₀ alkynyl, a branched C₄-C₁₀ alkynyl, a cyclic C₃-C₁₀ alkynyl, a linear C₁-C₆ alkynyl, a branched C₄-C₆ alkynyl, a cyclic C₃-C₆ alkynyl, a linear C₁-C₄ alkynyl, cyclic C₃-C₄ alkynyl, such as a linear C₁-C₁₀, C₁-C₉, C₁-C₈, C₁-C₇, C₁-C₆, C₁-C₅, C₁-C₄, C₁-C₃, C₁-C₂ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group.

It is understood that any of the exemplary alkyl, alkenyl, and alkynyl groups can be heteroalkyl, heteroalkenyl, and heteroalkynyl, respectively. For example, the alkyl can be a linear C₂-C₃₀ heteroalkyl, a branched C₄-C₃₀ heteroalkyl, a cyclic C₃-C₃₀ heteroalkyl (i.e. a heterocycloalkyl), a linear C₁-C₂₀ heteroalkyl, a branched C₄-C₂₀ heteroalkyl, a cyclic C₃-C₂₀ heteroalkyl, a linear C₁-C₁₀ heteroalkyl, a branched C₄-C₁₀ heteroalkyl, a cyclic C₃-C₁₀ heteroalkyl, a linear C₁-C₆ heteroalkyl, a branched C₄-C₆ heteroalkyl, a cyclic C₃-C₆ heteroalkyl, a linear C₁-C₄ heteroalkyl, cyclic C₃-C₄ heteroalkyl, such as a linear C₁-C₁₀, C₁-C₉, C₁-C₈, C₁-C₇, C₁-C₆, C₁-C₅, C₁-C₄, C₁-C₃, C₁-C₂ heteroalkyl group, a branched C₃-C₉, C₃-C₉, C₃-C₈, C₃-C₇, C₃-C₆, C₃-C₅, C₃-C₄ heteroalkyl group, or a cyclic C₃-C₉, C₃-C₉, C₃-C₈, C₃-C₇, C₃-C₆, C₃-C₅, C₃-C₄ heteroalkyl group.

For any of Formulae I′, I″, I″a, I″b, I″c, and I-III, the alkenyl can be a linear C₂-C₃₀ heteroalkenyl, a branched C₄-C₃₀ heteroalkenyl, a cyclic C₃-C₃₀ heteroalkenyl (i.e., a heterocycloalkenyl), a linear C₁-C₂₀ heteroalkenyl, a branched C₄-C₂₀ heteroalkenyl, a cyclic C₃-C₂₀ heteroalkenyl, a linear C₁-C₁₀ heteroalkenyl, a branched C₄-C₁₀ heteroalkenyl, a cyclic C₃-C₁₀ heteroalkenyl, a linear C₁-C₆ heteroalkenyl, a branched C₄-C₆ heteroalkenyl, a cyclic C₃-C₆ heteroalkenyl, a linear C₁-C₄ heteroalkenyl, cyclic C₃-C₄ heteroalkenyl, such as a linear C₁-C₁₀, C₁-C₉, C₁-C₈, C₁-C₇, C₁-C₆, C₁-C₅, C₁-C₄, C₁-C₃, C₁-C₂ heteroalkenyl group, a branched C₃-C₉, C₃-C₉, C₃-C₈, C₃-C₇, C₃-C₆, C₃-C₅, C₃-C₄ heteroalkenyl group, or a cyclic C₃-C₉, C₃-C₉, C₃-C₈, C₃-C₇, C₃-C₆, C₃-C₅, C₃-C₄ heteroalkenyl group.

For any of Formulae I′, I″, I″a, I″b, I″c, and I-III, the alkynyl can be a linear C₂-C₃₀ heteroalkynyl, a branched C₄-C₃₀ heteroalkynyl, a cyclic C₃-C₃₀ heteroalkynyl (i.e., a heterocycloalkynyl), a linear C₁-C₂₀ heteroalkynyl, a branched C₄-C₂₀ heteroalkynyl, a cyclic C₃-C₂₀ heteroalkynyl, a linear C₁-C₁₀ heteroalkynyl, a branched C₄-C₁₀ heteroalkynyl, a cyclic C₃-C₁₀ heteroalkynyl, a linear C₁-C₆ heteroalkynyl, a branched C₄-C₆ heteroalkynyl, a cyclic C₃-C₆ heteroalkynyl, a linear C₁-C₄ heteroalkynyl, cyclic C₃-C₄ heteroalkynyl, such as a linear C₁-C₁₀, C₁-C₉, C₁-C₈, C₁-C₇, C₁-C₆, C₁-C₅, C₁-C₄, C₁-C₃, C₁-C₂ heteroalkynyl group, a branched C₃-C₉, C₃-C₉, C₃-C₅, C₃-C₇, C₃-C₆, C₃-C₅, C₃-C₄ heteroalkynyl group, or a cyclic C₃-C₉, C₃-C₉, C₃-C₈, C₃-C₇, C₃-C₆, C₃-C₅, C₃-C₄ heteroalkynyl group.

For any of Formulae I′, I″, I″a, I″b, I″c, and I-III, the aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁1 heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. The polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

For any of Formulae I′, I″, I″a, I″b, I″c, and I-III, the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, or a thiol.

For any of Formulae I′, I″, I″a, I″b, I″c, and I-III, the substituents can be independently a substituted or unsubstituted alkyl, a substituted or unsubstituted aryl, a substituted or unsubstituted aralkyl, a carbonyl, an alkoxy, a halogen, a hydroxyl, a cyano, a nitro, an amino, an amido, or an oxo.

For any of Formulae I′, I″, I″a, I″b, I″c, and I-III, the substituents can be independently an unsubstituted alkyl (e.g. a linear C₁-C₆, C₁-C₅, C₁-C₄, C₁-C₃, C₁-C₂ alkyl group, a branched C₃-C₆, C₃-C₅, C₃-C₄ alkyl group, or a cyclic C₃-C₆, C₃-C₅, C₃-C₄ alkyl group), an unsubstituted aryl, an unsubstituted aralkyl, a carbonyl (e.g. a carboxyl), an alkoxy, a halogen, a hydroxyl, an amino, or an amido.

The prodrugs may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The prodrugs may be capable of existing as geometric isomers. Accordingly, it is to be understood that the disclosed prodrugs include pure geometric isomers or mixtures of geometric isomers.

### 2. Exemplary Prodrugs

An exemplary captopril-cinnamaldehyde (“CAPT-CA”) prodrug is presented below.

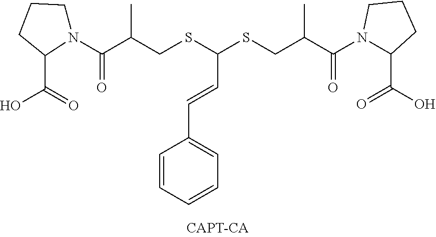 [SMILES: CC(CSC(/C=C/c1ccccc1)SCC(C)C(=O)N1CCCC1C(=O)O)C(=O)N1CCCC1C(=O)O]

CAPT and CA have anti-inflammatory properties and thus are therapeutic drugs for inflammatory diseases, such as RA. The sulfhydryl group of CAPT is unstable and undergoes oxidation to form captopril disulfide that induces adverse effects, such as transient taste loss, hypotension, and rashes. CA has poor bioavailability due to the rapid oxidation of its aldehyde group.

This exemplary CAPT-CA prodrug contains two CAPT moieties and one cinnamaldehyde moiety linked by a dithioacetal linking moiety, such that the active groups of these two active agents (i.e. the sulfhydryl group of CAPT and aldehyde group of CA) are protected from oxidation. Upon activation by ROS at an inflammatory site, the dithioacetal linking moiety of the CAPT-CA prodrug is cleaved, thereby release the active agents, two CAPT and one CA, in their free form as shown below, without any byproduct generation.

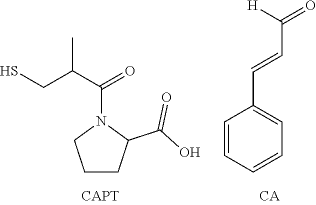

### 3. Pharmaceutically Acceptable Salts

The prodrugs may be neutral or may be one or more pharmaceutically acceptable salts, crystalline forms, non crystalline forms, hydrates, or solvates, or a combination thereof. References to the prodrugs may refer to the neutral molecule, and/or those additional forms thereof collectively and individually from the context. Pharmaceutically acceptable salts of the prodrugs include the acid addition and base salts thereof.

Suitable acid addition salts of the prodrugs are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the prodrugs are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases of the prodrugs may also be formed, for example, hemisulphate and hemicalcium salts.

### B. Pharmaceutical Compositions

Pharmaceutical compositions and pharmaceutical formulations in unit dosage form (also referred herein as “pharmaceutical formulations”) suitable for the delivery of the prodrugs (including their pharmaceutically acceptable salts thereof) and their preparation are disclosed. Generally, the pharmaceutical composition or formulation contains the prodrugs described herein and a pharmaceutically acceptable carrier and/or excipient. The term “pharmaceutically acceptable carrier” or “pharmaceutically acceptable excipient” is used herein to describe any ingredient in the formulation other than the prodrugs described herein. The pharmaceutical compositions or formulations can include an effective amount of one or more prodrugs of any of Formulae I′, I″, and I-III described herein and/or their pharmaceutically acceptable salts (together referred to as “prodrugs”), including any one or any combination of the prodrugs of Formulae I′, I″, and I-III described herein and/or their pharmaceutically acceptable salts, for preventing or treating an inflammatory disease or disorder, or treating or ameliorating one or more symptoms associated with an inflammatory disease or disorder in a subject in need thereof. It is to be understood that combinations and/or mixtures of the prodrugs may be included in the pharmaceutical composition or formulation.

In some forms, the pharmaceutical composition or formulation can further contain one or more active agents in their free forms in addition to the prodrugs, such as one or more additional anti-inflammatory agents.

Any one of more of the prodrugs provided herein can be expressly included or expressly excluded from the pharmaceutical compositions, dosage units, and/or methods of use or treatment disclosed herein.

### 1. Oral Formulations

The prodrugs can be administered orally. Oral administration may involve swallowing so that the prodrug enters the gastrointestinal tract, or buccal or sublingual administration may be employed by which the prodrug enters the bloodstream directly from the mouth.

Formulations suitable for oral administration of the prodrugs disclosed herein include solid formulations such as tablets, capsules containing particulates, liquids, powders, lozenges (including liquid-filled lozenges), chews, multi- and nano-particulates, gels, solid solutions, liposomes, films, ovules, sprays and liquid formulations. In some forms, the prodrugs can be associated with a suitable carrier, such as particles or micelles, e.g. polymeric particles or micelles, lipid particles, and dendrimers. In these forms, the prodrugs can be encapsulated in, covalently bond to, and/or complexed with the particles. These particles containing the prodrugs can be used for increasing the solubility of the prodrugs disclosed herein. Examples of materials suitable for forming the particles (nanoparticles and/or microparticles) containing the disclosed prodrugs include, but are not limited to, poly(alkylene glycol) or a copolymer thereof, such as poly(ethylene glycol) (“PEG”) and copolymers thereof, phospholipids, and polyamidoamine (“PAMAM”) or derivatives thereof (e.g. hydroxyl PAMAM). These particles containing the disclosed prodrugs may be further formulated into tablets, capsules, powders, etc.

Liquid formulations include suspensions, solutions, syrups and elixirs. Such formulations may be employed as fillers in soft or hard capsules and typically contain a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The prodrugs may also be used in fast dissolving, fast-disintegrating dosage forms such as those described in Expert Opinion in Therapeutic Patents, 11 (6), 981-986, by Liang and Chen (2001).

For tablet or capsule dosage forms, depending on dose, the prodrugs may make up from 1 weight % to 80 weight % of the dosage form, more typically from 5 weight % to 60 weight % of the dosage form. In addition to the prodrugs described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will contain from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may contain from 0.2 weight % to 5 weight % of the tablet, and glidants may contain from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally contain from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), antioxidants, colorants, flavouring agents, preservatives and taste-masking agents.

Exemplary tablets contain up to about 80% of one or more of the prodrugs described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations of the prodrugs for oral administration may be formulated to be immediate and/or modified release. Modified release formulations include delayed, sustained, pulsed, controlled, targeted and programmed release formulations.

### 2. Parenteral Formulations

The prodrugs can also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of the prodrugs used in the preparation of a parenteral formulation may be increased by the use of appropriate formulation techniques, such as the incorporation of solubility-enhancing agents or the association of the prodrugs with particles, such as those described above. For example, formulations for parenteral administration can contain a suitable carrier that can increase the solubility of the prodrugs disclosed herein. For example, the prodrugs disclosed herein can be encapsulated in, covalently bond to, or complexed with polymeric nanoparticles, microparticles, or micelles, such as nanoparticles, microparticles, or micelles formed by a poly(lactic-co-glycolic acid), poly(lactic-co-glycolic acid)-poly(ethylene glycol), poly(lactic acid)-poly(ethylene oxide), poly(caprolactone)-poly(ethylene glycol), or a copolymer thereof.

Formulations of the prodrugs for parenteral administration may be formulated to be immediate and/or modified release. Modified release formulations include delayed, sustained, pulsed, controlled, targeted and programmed release formulations. Thus, the prodrugs may be formulated as a solid, semi-solid, or thixotropic liquid for administration as an implanted depot providing modified release of the active agents. Examples of such formulations include drug-coated stents and poly(dl-lactic-coglycolic)acid (PGLA) microspheres.

### 3. Pulmonary and Mucosal Formulations

The prodrugs can be formulated for pulmonary or mucosal administration. The administration can include delivery of the composition to the lungs, nasal, oral (sublingual, buccal), vaginal, or rectal mucosa.

For example, the prodrugs can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may contain a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebulizer contains a solution or suspension of one or more of the prodrugs including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the prodrugs is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the prodrugs described herein, a suitable powder base such as lactose or starch and a performance modifier such as 1-leucine, mannitol, or magnesium stearate. The lactose may be anhydrous or in the form of a monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the prodrugs described herein per actuation and the actuation volume may vary from 1 μl to 100 μl. A typical formulation may contain one or more of the prodrugs described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the prodrugs are typically arranged to administer a metered dose or “puff”. The overall daily dose will be administered in a single dose or, more usually, as divided doses throughout the day.

In some forms, the prodrugs described herein can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the prodrugs. An appropriate solvent should be used that dissolves the prodrugs or forms a suspension of the prodrugs. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical compositions may contain minor amounts of polymers, surfactants, or other excipients well known to those of the art. In this context, “minor amounts” means no excipients are present that might affect or mediate uptake of the prodrugs by cells and that the excipients that are present in amount that do not adversely affect uptake of prodrugs by cells. In some forms, the prodrugs described herein may be associated with particles, such as those described above. For example, formulations for pulmonary or mucosal administration can contain a suitable carrier that can increase the solubility of the prodrugs disclosed herein. For example, the prodrugs disclosed herein can be encapsulated in, covalently bond to, or complexed with polymeric nanoparticles, microparticles, or micelles, such as nanoparticles, microparticles, or micelles formed by a poly(alkylene glycol) or a copolymer thereof.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

### 4. Topical Formulations

The prodrugs can be administered directly to the external surface of the skin or the mucous membranes (including the surface membranes of the nose, lungs and mouth), such that the prodrugs cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations for topical administration may be formulated to be immediate and/or modified release. Modified release formulations include delayed, sustained, pulsed, controlled, targeted and programmed release formulations. Thus, the prodrugs may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### 5. Additional Active Agent(s)

In some forms, the pharmaceutical composition or pharmaceutical formulation can include one or more additional active agents in their free form, such as one or more additional anti-inflammatory agents. Anti-inflammatory agents that can be included in the pharmaceutical compositions or formulations are known, for example, see the WebMD, “Anti-inflammatory Drugs,” web site webmd.com/arthritis/anti-inflammatory-drugs; Barnes, Nature, 402(6760): 31-38 (1999); and Rainsford, Inflammation in the pathogenesis of chronic diseases 3:27 (2007).

Exemplary anti-inflammatory drugs that can be included in the pharmaceutical composition or pharmaceutical formulation include, but are not limited to, ibuprofen, naproxen sodium, aspirin, naproxen sodium, diclofenac potassium, celecoxib, sulindac, oxaprozin, piroxicam, indomethacin, meloxicam, fenoprofen, naproxen, esomeprazole, diclofenac, diflunisal, etodolac, ketorolac tromethamine, katoprofen, meclofenamate, nabumetone, salsalate, tolmetin, and steroids, such as corticosteroids (e.g. hydrocortisone, cortisone, ethamethasoneb, prednisone, prednisolone, triamcinolone, methylprednisolone, and dexamethasone) and mineralocorticoids (e.g. fludrocortisone), and a combination thereof.

### 6. Effective Amount

Effective amounts of the prodrugs contained in the pharmaceutical composition or pharmaceutical formulation depend on many factors, including the indication being treated, the route of administration, co-administration of other therapeutic compositions, and the overall condition of the patient. Exemplary effective amount of the prodrugs contained in the pharmaceutical formulation (in unit dosage form) can be from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg.

In some forms, the total concentration of the one or more prodrugs in the pharmaceutical formulation can be at least 0.01 wt %, at least 0.05 wt %, at least 0.1 wt %, in a range from about 0.01 wt % to about 50 wt %, from about 0.05 wt % to about 50 wt %, from about 0.1 wt % to about 50 wt %, from about 0.01 wt % to about 40 wt %, from about 0.05 wt % to about 40 wt %, from about 0.1 wt % to about 40 wt %, from about 0.01 wt % to about 30 wt %, from about 0.05 wt % to about 30 wt %, from about 0.1 wt % to about 30 wt %, from 0.01 wt % to 20 wt %, from about 0.05 wt % to about 20 wt %, from about 0.1 wt % to about 20 wt %, from about 0.1 wt % to about 15 wt %, from about 0.2 wt % to about 20 wt %, from about 0.1 wt % to about 10 wt %, from about 0.5 wt % to about 20 wt %, from about 0.5 wt % to about 15 wt %, from about 0.5 wt % to about 10 wt %, from about 0.5 wt % to about 5 wt %, from about 0.1 wt % to about 5 wt %, or from about 0.1 wt % to about 1 wt %. The term “total concentration of the one or more prodrugs in the pharmaceutical formulation” refers to the sum of the weight of the one or more prodrugs relative to the total weight of the pharmaceutical formulation.

### 7. Exemplary Pharmaceutical Formulations

An exemplary pharmaceutical formulation of the prodrugs disclosed herein includes polymeric particles or micelles, such as particles or micelles formed by poly(ethylene glycol), poly(lactic-co-glycolic acid), poly(lactic-co-glycolic acid)-poly(ethylene glycol), poly(lactic acid)-poly(ethylene oxide), poly(caprolactone)-poly(ethylene glycol), or copolymers thereof, as the carrier and one or more of the prodrugs disclosed herein; the prodrugs are encapsulated in, conjugated to, or complexed with the polymeric particles or micelles. The polymeric particles or micelles containing the prodrugs may be administered in such a solid form or combined with one or more pharmaceutically acceptable excipients and further formulated into tablets, capsules, powders, etc. for administration. The polymeric particles or micelles containing the prodrugs may be reconstituted into a liquid form prior to administration, such as for oral administration and/or parenteral administration.

### III. Methods of Making

Methods of making the prodrugs are disclosed. Generally, the method includes (i) cooling a reaction mixture at a suitable temperature for a period of time sufficient to form a product containing one or more of the prodrugs of any one of Formulae I′, II″, and I-III. In some forms, the method involves only a one-step reaction and produces the prodrugs at a yield of at least about 50 wt %, at least about 60 wt %, or at least about 70 wt %. The yield of the prodrug can be calculated using the formula: % yield=(actual weight of the purified prodrug/theoretical weight of the prodrug)×100%.

The reaction mixture contains at least two reactants and a solvent. Suitable solvents for forming the reactant mixture depend on the solubility of the reactants. Typically, the solvent can dissolve all of the reactants and form a solution. Examples of suitable solvents for forming the reaction mixture include, but are not limited to, dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof.

In some forms, the reaction mixture can further contain a catalyst. Examples of suitable catalyst that can be used in the reaction include, but are not limited to, trifluoroacetic acid, p-toluene sulfonic acid, and boron trifluoride, and a combination thereof.

In some forms, the reaction mixture contains a first reactant, a second reactant, and a third reactant, where the first reactant can have the structure of Formula Ia′:

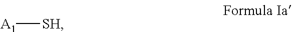

the second reactant can have the structure of Formula Ib′:

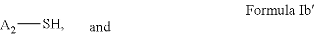

the third reactant can have the structure of Formula Ic′:

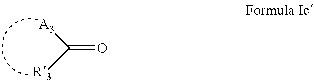

where each of A₁, A₂, and A₃ can be an active agent moiety; R′₃ can be absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; [symbol] can be independently absent or a bond (single, double, or triple); and the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

In some forms, the first and second reactants can be independently penicillamine or a penicillamine derivative, tiopronin or a tiopronin derivative, methimazole or a methimazole derivative, omapatrilat or an omapatrilat derivative, or captopril or a captopril derivative. In some forms, A₁ of Formula Ia′ can be a moiety of

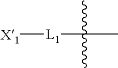

and A₂ of Formula Ib′ can be a moiety of

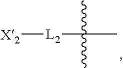

where L₁ and L₂ can be independently absent, a bond (single, double, or triple), a substituted or unsubstituted alkyl, a substituted or unsubstituted alkenyl, an amino, an amido, a carbonyl, an alkoxyl, a polyether, a thioether; and X′₁ and X′₂ can be independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl). In some forms, X′₁-L₁- and X′₂-L₂- can be independently a moiety of Formula I″b as shown and defined above. In some forms, X′₁-L₁- and X′₂-L₂- can be independently a moiety of Formula I″c as shown and defined above. In some forms, X′₁-L₁- and X′₂-L₂- can be independently:

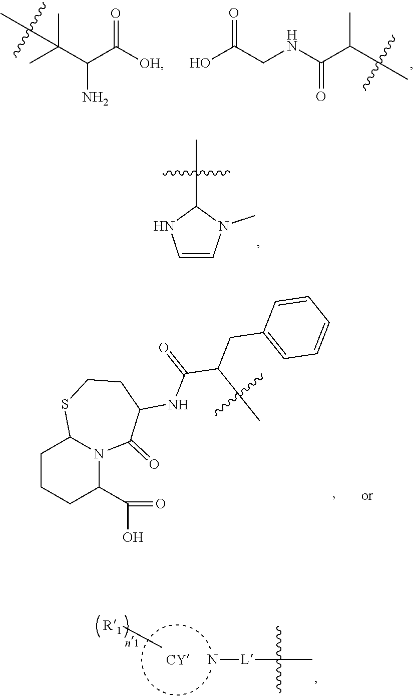

where: (a) L′ can be a bond (single, double, or triple), a substituted or unsubstituted alkyl, a substituted or unsubstituted alkenyl, an amino, an amido, a carbonyl, an alkoxyl, a polyether, a thioether; (b) CY′ can be independently a substituted or unsubstituted heteroaryl, a substituted or unsubstituted polyheteroaryl, or a substituted or unsubstituted heterocyclyl; (c) R′₁ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether; and (d) n′1 can be an integer from 0 to 10. In some forms, the reaction mixture contains a first reactant and a second reactant, where the first reactant can have the structure of Formula Id′:

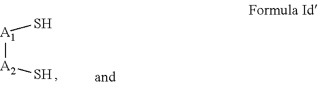

where A₁ and A₂ can be an active agent moiety and the second reactant can have the structure of Formula Ic′ as described above.

In some forms, the reaction mixture contains a first reactant, a second reactant, and a third reactant, where each of the first and second reactants can be captopril or a captopril derivative and the third reactant can be a cinnamaldehyde or a cinnamaldehyde derivative. In some forms, the first reactant that is a captopril or a captopril derivative can have the same structure as or a structure that is different from the second reactant that is a captopril or a captopril derivative.

In some forms, the reaction mixture contains a first reactant having the structure of Formula Ia, a second reactant having the structure of Formula Ib, and a third reactant having the structure of Formula Ic:

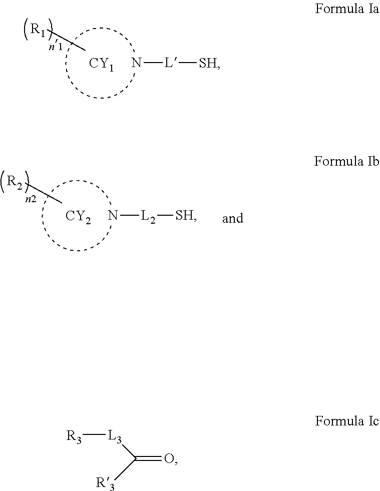

where: (a) L₁, L₂, and L₃ can be independently a bond (single, double, or triple), a substituted or unsubstituted alkyl, a substituted or unsubstituted alkenyl, an amino, an amido, a carbonyl, an alkoxyl, a polyether, a thioether; (b) CY₁ and CY₂ can be independently a substituted or unsubstituted heteroaryl, a substituted or unsubstituted polyheteroaryl, or a substituted or unsubstituted heterocyclyl; (c) R₁ and R₂ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether; (d) n1 and n2 can be independently an integer from 0 to 10; (e) R₃ can be a substituted or unsubstituted aryl, a substituted or unsubstituted polyaryl, a substituted or unsubstituted heteroaryl, or a substituted or unsubstituted polyheteroaryl; (f) R′₃ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and (g) the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

In some forms, the first reactant of Formula Ia can be the same as or different from the second reactant of Formula Ib. In some forms, the first reactant of Formula Ia can be the same as the second reactant of Formula Ib, i.e. L₁ is the same as L₂, CY₁ is the same as CY₂, R₁ is the same as R₂, and n1 is the same as n2. In some forms, the first reactant of Formula Ia can be different from the second reactant of Formula Ib, i.e. at least one of the pairs: L₁ and L₂, CY₁ and CY₂, R₁ and R₂, and n1 and n2, is different.

In some forms, the reaction mixture contains a first reactant having the structure of Formula IIa, a second reactant having the structure of Formula IIb, and a third reactant having the structure of Formula IIc:

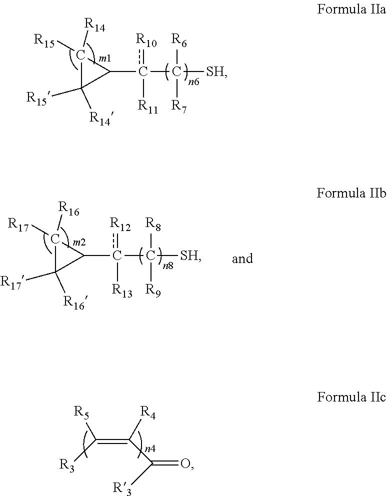

where: (a) each occurrence of R₄-R₉, R₁₄-R₁₇, and R₁₄′-R₁₇′ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (b) m1 and m2 can be independently an integer from 1 to 9; (c) n4, n6, and n8 can be independently an integer from 1 to 10; (d) R₁₀ and R₁₂ can be independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (e) R₁₁ and R₁₃ can be independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol; (f) R′₃, R₃, and the substituents can be as defined above for Formulae Ia-Ic.

In some forms, the first reactant of Formula IIa can be the same as or different from the second reactant of Formula IIb. In some forms, the first reactant of Formula IIa can be the same as the second reactant of Formula IIb. In some forms, the first reactant of Formula IIa can be different from the second reactant of Formula IIb.

In some forms, the reaction mixture contains a first reactant having the structure of Formula IIIa, a second reactant having the structure of Formula IIIb, and a third reactant having the structure of Formula IIc (as shown above):

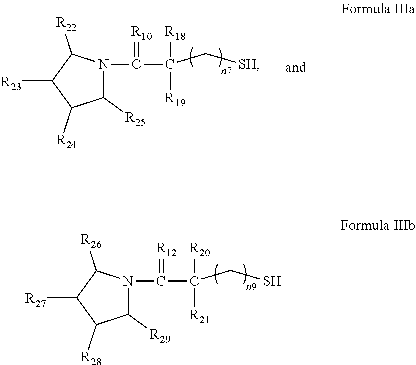

where: (a) n7 and n9 can be independently an integer from 0 to 8; (b) R₁₈, R₁₉, R₂₀, and R₂₁ can be independently hydrogen, a substituted or unsubstituted alkyl, a substituted or unsubstituted aryl, an alkoxyl, a hydroxyl, or a halogen; (c) R₁₀ and R₁₂ can be independently oxygen or sulfur; (d) R₂₂-R₂₉ can be independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol; and the substituents can be as defined above for Formulae Ia-Ic.

In some forms, the first reactant of Formula IIIa can be the same as or different from the second reactant of Formula IIIb. In some forms, the first reactant of Formula IIIa can be the same as the second reactant of Formula IIIb. In some forms, the first reactant of Formula IIIa can be different from the second reactant of Formula IIIb.

In some forms of Formulae IIIa and IIIb, at least one of R₁₈ and R₁₉ and/or at least one of R₂₀ and R₂₁ can be a substituted or unsubstituted alkyl, a substituted or unsubstituted aryl, an alkoxyl, a hydroxyl, or a halogen. In some forms of Formulae IIIa and IIIb, at least one of R₁₈ and R₁₉ and/or at least one of R₂₀ and R₂₁ is a substituted or unsubstituted alkyl. In some forms of Formulae IIIa and IIIb, at least one of R₂₂ and R₂₅ and/or at least one of R₂₆ and R₂₈ is

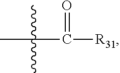

and R₃₁ is a hydroxyl, an alkoxyl, or an amino.

In some forms of Formula IIc, R₄ and R₅ can be independently hydrogen, a substituted or unsubstituted alkyl, a substituted or unsubstituted aryl, an alkoxyl, a hydroxyl, or a halogen.

In some forms of Formulae Ic and IIc, R₃ can be

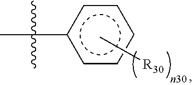

n30 can be an integer from 0 to 5, and R₃₀ can be hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether.

In some forms, the first and second reactant in the reaction mixture are captopril (“CAPT”) and the third reactant is cinnamaldehyde (“CA”), having the structures shown below:

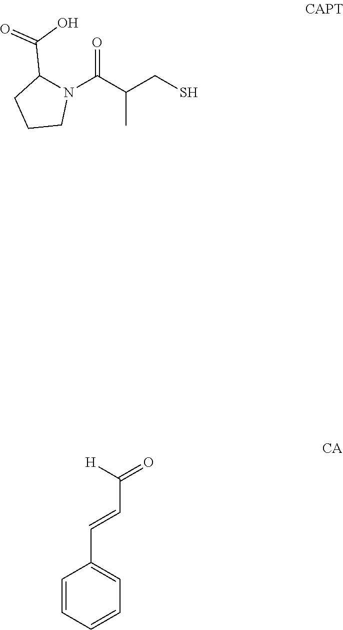

The reaction mixture containing the reactants and optionally the catalyst in the solvent can be cooled at a suitable temperature for a period of time sufficient to form a product containing one or more prodrugs having the structures of any one of Formulae I′, I″, and I-III. In some forms, the reaction mixture can be cooled at a temperature in a range from 0° C. to −4° C., for a time period in a range from 12 hour to 36 hours.

In some forms, the method can further include a step of mixing the reactants and the solvent and optionally the catalyst to form the reaction mixture prior to step (i). In some forms, the method can further include a step of purifying the product containing the one or more prodrugs, such as prodrugs having the structures of any one of Formulae I′, I″, I″a, and I-III. In some forms, the product containing the compound can be purified by column chromatography using a suitable organic solvent as eluent, such as ethyl acetate or hexane, or a combination thereof. In some forms, following purification, the prodrugs is produced in the form of a solid.

In some forms, the method can further include one or more of the above-described steps: mixing the reactants and the solvent and optionally the catalyst to form the reaction mixture prior to step (i) and/or purifying the product containing the one or more prodrugs subsequent to step (i).

More specific methods for synthesizing the exemplary prodrug are described in the Examples below.

### IV. Methods of Use

Methods of using the prodrugs are described herein. The disclosed prodrugs can be used for preventing or treating a variety of diseases or disorders, or treating or ameliorating one or more symptoms associated with a variety of diseases or disorders, such as inflammatory diseases or disorders, cancers, and hypertension.

A. Preventing or Treating or Ameliorating Symptom(s) Associated with Inflammatory Diseases or Disorders

Methods of using the prodrugs for preventing or treating an inflammatory disease or disorder, or treating or ameliorating one or more symptoms associated with an inflammatory disease or disorder in a subject in need thereof are disclosed. The inflammatory disease or disorder can be local or systemic in the subject.

Generally, the method for preventing or treating an inflammatory disease or disorder, or treating or ameliorating one or more symptoms associated with an inflammatory disease or disorder in a subject in need thereof includes administering to the subject a pharmaceutical formulation containing one or more of the prodrugs disclosed herein. The pharmaceutical formulation can be administered in an effective amount to prevent or treat the inflammatory disease or disorder, or treat or ameliorate one or more symptoms associated with the inflammatory disease or disorder in the subject, as shown by one or more known clinical and/or biochemical measurements, such as Ritchie articular index (AI), early-morning stiffness, clinical score, grip strength, pain score, supine blood pressure, hemoglobin (Hb), white blood cell count, platelets, immunoglobulin M, plasma viscosity, C-reactive protein, or histidine, or a combination thereof (Martin, et al., “Captopril: a new treatment for rheumatoid arthritis?” The Lancet, 1984).

In some forms, the pharmaceutical formulation can be administered in an effective amount to change one or more clinical measurements of the subject, such as to decrease the Ritchie articular index (AI), decrease early-morning stiffness, increase clinical score, increase grip strength, and/or decrease pain score of the subject, compared to the subject before administered with the pharmaceutical formulation. For example, the pharmaceutical formulation can be administered in an effective amount to decrease the Ritchie articular index (AI) by at least 20%, at least 25%, or at least 30%; to decrease early-morning stiffness by at least 30%, at least 40%, at least 50%, at least 60%, at least 70%, at least 80%, or at least 90%; to increase clinical score by at least 5%, at least 8%, or at least 10%; to increase grip strength by at least 30%, at least 40%, at least 50%, or at least 60%; and/or to decrease pain score by at least 10%, at least 20%, or at least 30% of the subject, compared to the subject before administered with the pharmaceutical formulation. In some forms, the change of the one more clinical measurements described above can occur within 1 week, within 2 weeks, within 4 weeks, within 8 weeks, within 12 weeks, within 24 weeks, within 36 weeks, or within 48 weeks.

In some forms, the pharmaceutical formulation can be administered in an effective amount to change one or more biochemical measurements of the subject, such as to increase the hemoglobin (Hb) level, decrease the immunoglobulin M level, increase the serum sulphydryl level, decrease the plasma viscosity, decrease the C-reactive protein level, and/or increase the histidine level in the blood of the subject compared to the blood of the subject before administered with the pharmaceutical formulation. For example, the pharmaceutical formulation can be administered in an effective amount to increase the hemoglobin (Hb) level by at least 5% or at least 10%; to decrease the immunoglobulin M level by at least 10%, at least 15%, at least 20%, or at least 25%; to increase the serum sulphydryl level by at least 10%, at least 15%, at least 20%, or at least 25%; to decrease the plasma viscosity by at least 5% or at least 8%; to decrease the C-reactive protein level by at least 50%, at least 60%, at least 70%, or at least 80%; and/or to increase the histidine level by at least 20%, at least 30%, or at least 40%, in the blood of the subject, compared to the blood of the subject before administered with the pharmaceutical formulation. In some forms, the change of the one more biochemical measurements described above can occur within 1 week, within 2 weeks, within 4 weeks, within 8 weeks, within 12 weeks, within 24 weeks, within 36 weeks, or within 48 weeks.

In some forms, the pharmaceutical formulation can be administered in an effective amount to change one or more clinical and one or more biochemical measurements of the subject as described above. For example, the pharmaceutical formulation can be administered in an effective amount to decrease the Ritchie articular index (AI) by at least 20%, at least 25%, or at least 30%; to decrease early-morning stiffness by at least 30%, at least 40%, at least 50%, at least 60%, at least 70%, at least 80%, or at least 90%; to increase clinical score by at least 5%, at least 8%, or at least 10%; to increase grip strength by at least 30%, at least 40%, at least 50%, or at least 60%; and/or to decrease pain score by at least 10%, at least 20%, or at least 30% of the subject, compared to the subject before administered with the pharmaceutical formulation, and to increase the hemoglobin (Hb) level by at least 5% or at least 10%; to decrease the immunoglobulin M level by at least 10%, at least 15%, at least 20%, or at least 25%; to increase the serum sulphydryl level by at least 10%, at least 15%, at least 20%, or at least 25%; to decrease the plasma viscosity by at least 5% or at least 8%; to decrease the C-reactive protein level by at least 50%, at least 60%, at least 70%, or at least 80%; and/or to increase the histidine level by at least 20%, at least 30%, or at least 40% in the blood of the subject, compared to the blood of the subject before administered with the pharmaceutical formulation, where the change of the one more clinical and one or more biological measurements of the subject occurs within 1 week, within 2 weeks, within 4 weeks, within 8 weeks, within 12 weeks, within 24 weeks, within 36 weeks, or within 48 weeks.

The step of administering an effective amount of the pharmaceutical formulation can be achieved in a single administration step or using multiple steps of administering the pharmaceutical formulation. The subject can be a mammal, such as a human, a dog, a cat, a rat, a monkey, rabbits, guinea pigs, etc., that is in need of treatment.

The prodrugs disclosed herein contains active agent moieties linked by a ROS-responsive linking moiety. Due to the abundant ROS generated at an inflammatory site, the linking moiety of the prodrug can be cleaved by the ROS. Upon administration of the pharmaceutical formulation containing the disclosed prodrugs, the prodrugs can be cleaved by the ROS at an inflammatory site and release two or more active agents in their free form, without any byproduct generation. For example, the thioacetal linking moiety of the prodrug of Formula II or III can be cleaved by ROS, such as H₂O₂, at an inflammatory site and release active agents of IIa, IIb, and IIc, or IIIa, IIIb, and IIc. These prodrugs can increase the stability and bioavailability of the active agents and reduce the side effects of these active agents, compared with the same active agents administered in their free form.

### 1. Diseases

Both acute and chronic inflammatory diseases or disorders can be treated by the disclosed methods. Examples of suitable inflammatory diseases or disorders and symptoms associated with the inflammatory diseases or disorders that can be treated by the disclosed method include, but are not limited to, asthma, chronic peptic ulcer, tuberculosis, rheumatoid arthritis, periodontitis, ulcerative colitis, Crohn's disease, sinusitis, active hepatitis, acute bronchitis, appendicitis, ingrown toenail, sore throat, and physical trauma or wound, and a combination thereof.

In some forms, the inflammatory disease or disorder being treated by the disclosed method is rheumatoid arthritis.

### 2. Administration Routes

The pharmaceutical formulation containing one or more of the disclosed prodrugs can be administered to the subject by oral administration, parenteral administration, inhalation, mucosal, topical administration, or a combination thereof.

For example, the pharmaceutical formulation containing one or more of the disclosed prodrugs can be orally administered to a subject by a medical professional or the subject being treated (e.g., self-administration). The pharmaceutical formulation containing one or more of the disclosed prodrugs can be administered as tablets, capsules containing particulates, granules, powders, lozenges (including liquid-filled lozenges), chews, multi- and nano-particulates, gels, or liquids (e.g., solution or suspensions in aqueous or non-aqueous solvent).

Optionally, the pharmaceutical formulation containing one or more of the disclosed prodrugs can be administered to the subject by intravenous injection or intraperitoneal injection. The intravenous injection or intraperitoneal injection can be performed by a medical professional or the subject being treated (e.g., self-injection).

Alternatively, the pharmaceutical formulation containing one or more of the disclosed prodrugs can be administered to the subject by inhalation, such as mouth inhalation and/or nasal inhalation.

Optionally, the pharmaceutical formulation containing one or more of the disclosed prodrugs can be administered to the subject by topically applying the compound(s) or the pharmaceutical composition or formulation on one or more of the exposed surfaces of the subject.

### 3. Effective Amount

The step of administering an effective amount of the pharmaceutical formulation can be achieved in a single administration step or using multiple steps of administering the pharmaceutical formulation. For example, if the unit dosage form contains an effective amount of the prodrugs to prevent or treat the inflammatory disease or disorder, or treat or ameliorate one or more symptoms associated with the inflammatory disease or disorder in the subject is administered to the subject, as shown by one or more clinical measurements and/or one or more biochemical measurements of the subject as described above, then the method only requires a single administration step. Alternatively, if the unit dosage form contains less than the needed effective amount of the prodrugs to prevent or treat the inflammatory disease or disorder, or treat or ameliorate one or more symptoms associated with the inflammatory disease or disorder in the subject is administered to the subject, as shown by one or more clinical measurements and/or one or more biochemical measurements of the subject as described above, then the method involves at least two steps of administering the pharmaceutical formulation, and optionally more than two steps of administering the pharmaceutical formulation to the subject until an effective amount of the pharmaceutical formulation is administered to the subject to prevent or treat the inflammatory disease or disorder, or treat or ameliorate one or more symptoms associated with the inflammatory disease or disorder in the subject is administered to the subject, as shown by one or more clinical measurements and/or one more more biochemical measurements of the subject as described above. When multiple administration steps are needed to administer an effective amount of the pharmaceutical formulation to the subject, each administration step may involve administering the same dosage or different dosages of the pharmaceutical formulation to the patient. In some forms, the method involves a single administration of the pharmaceutical formulation in an effective amount to prevent or treat the inflammatory disease or disorder, or treat or ameliorate one or more symptoms associated with the inflammatory disease or disorder in the subject is administered to the subject, as shown by one or more clinical measurements and/or one or more biochemical measurements of the subject as described above.

In some forms, the pharmaceutical formulation is administered to the subject in an effective amount to prevent or treat the inflammatory disease or disorder, or treat or ameliorate one or more symptoms associated with the inflammatory disease or disorder in the subject is administered to the subject, as shown by one or more clinical measurements and/or one or more biochemical measurements of the subject as described above, without one or more side effects, such as skin rash, nephropathy, dizziness, mouth sores, a burning or itching sensation, or white patches in the mouth, or a combination thereof (Huang, et al., International Journal of Pharmaceutics, 241:345-351 (2002); website healthline.com/nutrition/side-effects-of-cinnamon #2.-May-Increase-the-Risk-of-Cancer).

In some forms, treatment regimens utilizing the prodrugs include administration of from about 0.1 mg to about 300 mg of the prodrugs per kilogram body weight of the recipient per day in multiple doses or in a single dose. In some embodiments, a suitable dose may be in the range of 0.1 to 300 mg per kilogram body weight of the recipient per day, optionally in the range of 6 to 150 mg per kilogram body weight per day, optionally in the range of 15 to 100 mg per kilogram body weight per day, optionally in the range of 15 to 80 mg per kilogram body weight per day, optionally in the range of 15 to 50 mg per kilogram body weight per day, and optionally in the range of 15 to 30 mg per kilogram body weight per day.

The desired dose may be presented as two, three, four, five or six or more sub-doses administered at appropriate intervals throughout the day. These sub-doses may be administered in unit dosage forms, for example, containing from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg of the prodrugs per unit dosage form.

### 4. Optional Steps

a. Administering Additional Active Agent(s)

One or more active agents may be administered to the subject in their free forms throughout the method or at different intervals during the method. For example, the one or more additional active agents in their free forms is administered to the subject prior to, during, and/or subsequent to step (i) administering the pharmaceutical formulation containing the prodrugs. In some forms, the one or more additional active agents in their free form can be included in the pharmaceutical formulation containing the prodrug(s) and administered to the subject simultaneously with the prodrug(s).

In some forms, the one or more additional active agents are one or more anti-inflammatory agents as described above. The amount of the one or more additional anti-inflammatory agents administered will vary from subject to the subject according to their need.

### B. Treating Macrophages

In some forms, the prodrugs can be used in a method for treating macrophages in a subject in need thereof.

The method can follow the method step described above, for example, administering to the subject an effective amount of a pharmaceutical formulation containing one or more of the prodrugs by oral administration, parenteral administration, inhalation, mucosal, topical administration, or a combination thereof. In some forms, the method can include the additional step described above. For example, the user can administer one or more additional active agents in their free form to the subject prior to, during, and/or subsequent to administering the pharmaceutical formulation to the subject.

In some forms of the method, the prodrugs can reduce the production of nitric oxide by macrophages in the subject, compared with the production of nitric oxide by the macrophages in a control not treated with the prodrugs, tested under the same conditions.

Additionally or alternatively, the prodrugs can reduce the release of proinflammatory cytokines from the macrophages in the subject, compared with the release of the proinflammatory cytokines from the macrophages in a control not treated with the prodrugs, tested under the same conditions.

In some forms of the method, the prodrugs can reduce the production of nitric oxide by macrophages without affecting the cell viability of the macrophages in the subject, compared with the production of nitric oxide by the macrophages in a control not treated with the prodrugs, tested under the same conditions. Additionally or alternatively, the prodrugs can reduce the release of proinflammatory cytokines by macrophages without affecting the cell viability of the macrophages in the subject, compared with the release of the proinflammatory cytokines by the macrophages in a control not treated with the prodrugs, tested under the same conditions.

### 1. Reduction of Nitric Oxide Production

Nitric oxide (“NO”) is a marker of inflammatory responses. Measuring the production of NO can be used for evaluating the progression of inflammation; inhibition of its production can be used for controlling inflammatory reactions and diseases.

In some forms of the method, the prodrugs can reduce production of nitric oxide by the macrophages in the subject, compared with the production of nitric oxide by the macrophages in a control not treated with the prodrugs, tested under the same conditions. “Same conditions” for testing the production of nitric oxide means the test is performed using the same assay and using the same protocol, such as the same amount of cells and enzymes, same buffer and buffer concentration, same dye and dye concentration, and same incubation time and temperature, etc.

In some forms of the method, the pharmaceutical formulation administered to the subject is in an effective amount to reduce production of nitric oxide by the macrophages in the subject, compared with the production of nitric oxide by the macrophages in a control not treated with the prodrugs, tested under the same conditions.

In some forms of the method, the pharmaceutical formulation is administered to the subject in an effective amount to reduce the production of NO by the macrophages in the subject by at least 20%, at least 30%, at least 40%, at least 45%, at least 50%, at least 55%, at least 60%, at least 65%, at least 70%, at least 75%, at least 80%, at least 85%, at least 90%, in a range from 20% to 95%, from 20% to 90%, from 20% to 85%, from 30% to 80%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in a control not treated with the prodrugs, tested under the same conditions.

In some forms of the method, the pharmaceutical formulation is administered to the subject in an effective amount to reduce the production of NO by the macrophages without affecting cell viability of the macrophages in the subject in the subject by at least 20%, at least 30%, at least 40%, at least 45%, at least 50%, at least 55%, at least 60%, at least 65%, at least 70%, at least 75%, at least 80%, at least 85%, at least 90%, in a range from 20% to 95%, from 20% to 90%, from 20% to 85%, from 30% to 80%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in a control not treated with the prodrugs, tested under the same conditions. The term “without affecting cell viability of the macrophages” means that the reduction of cell viability of the macrophages treated with the prodrugs is less than about 20%, less than about 15%, less than about 10%, or less than about 5% compared with the cell viability of macrophages not treated with the prodrugs in a 24-h period.

### 2. Reduction of Proinflammatory Cytokine Release

Macrophages can release proinflammatory cytokines, such as IL-10, IL-6, IL-10, and TNF-α, which participate in the pathogenesis of inflammatory conditions.

In some forms of the method, the prodrugs can reduce the amount of an proinflammatory cytokine, such as IL-10, IL-6, IL-10, and/or TNF-α, released from macrophages of the subject, compared with the amount of an proinflammatory cytokine released from the macrophages in a control not treated with the prodrugs, tested under the same conditions. “Same conditions” for testing the amount of proinflammatory cytokine released from macrophages means test is performed using the same assay and using the same protocol, such as same amount of cells and enzymes, same buffer and buffer concentration, same dye and dye concentration, and same incubation time and temperature, etc.

In some forms of the method, the pharmaceutical formulation is administered to the subject in an effective amount to reduce the amount of an proinflammatory cytokine, such as IL-1β, IL-6, IL-10, and/or TNF-α, released from macrophages in the subject, compared with the amount of the proinflammatory cytokine released from the macrophages in a control not treated with the prodrugs, tested under the same conditions.

In some forms of the method, the pharmaceutical formulation is administered to the subject in an effective amount to reduce the amount of an proinflammatory cytokine, such as IL-1β, IL-6, IL-10, and/or TNF-α, released from macrophages in the subject as shown by a reduced protein and/or mRNA level of the proinflammatory cytokine, compared with the protein and/or mRNA level of the proinflammatory cytokine released from the macrophages in a control not treated with the prodrugs, tested under the same conditions.

In some forms of the method, the pharmaceutical formulation is administered to the subject in an effective amount to reduce the amount of an proinflammatory cytokine, such as IL-1β, IL-6, IL-10, and/or TNF-α, released from macrophages in the subject, as shown by a reduced protein level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, and/or a reduced mRNA level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, compared with the protein and/or mRNA levels of the proinflammatory cytokine released from the macrophages in a control not treated with the prodrugs, tested under the same conditions.

In some forms of the method, the pharmaceutical formulation is administered to the subject in an effective amount to reduce the amount of an proinflammatory cytokine, such as IL-10, IL-6, IL-10, and/or TNF-α, released from macrophages without affecting the cell viability of the macrophages in the subject, as shown by a reduced protein level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, and/or a reduced mRNA level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, compared with the protein and/or mRNA levels of the proinflammatory cytokine released from the macrophages in a control not treated with the prodrugs, tested under the same conditions.

In some forms, the pharmaceutical formulation is administered to the subject in an effective amount to reduce the production of NO by the macrophages in the subject by at least 20%, at least 30%, at least 40%, at least 45%, at least 50%, at least 55%, at least 60%, at least 65%, at least 70%, at least 75%, at least 80%, at least 85%, at least 90%, in a range from 20% to 95%, from 20% to 90%, from 20% to 85%, from 30% to 80%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in a control not treated with the prodrugs, tested under the same conditions; and to reduce the amount of an proinflammatory cytokine, such as IL-10, IL-6, IL-10, and/or TNF-α, released from macrophages in the subject, as shown by a reduced protein level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, and/or a reduced mRNA level of the proinflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, compared with the protein and/or mRNA levels of the proinflammatory cytokine released from the macrophages in the control not treated with the prodrugs, tested under the same conditions.

C. Treating or Ameliorating Symptom(s) Associated with Cancer and/or Hypertension

In some forms, the prodrugs can be used in a method for treating or ameliorating one or more symptoms associated with a cancer in a subject in need thereof.

In some forms, the prodrugs can be used in a method for treating or ameliorating one or more symptoms associated with hypertension in a subject in need thereof.

The microenvironment of cancer cells or hypertension patients produces excessive amounts of ROS (Liou and Storz, Free Radic Res. 2010, 44, 479; Togliatto, et al., Int J Mol Sci. 2017, 18, 1988; El-Bassossy, et al., Food Chem Toxicol. 2011, 49, 3007; Liu, et al., Front, Pharmacol. 2020, 11, 582719; and Captopril, anti-hypertension, cancer treatment. WIKIPEDIA). Upon administration of the pharmaceutical formulation that contains the prodrugs disclosed herein into a subject having cancer and/or hypertension, the thioacetal linking moiety of the prodrugs can be cleaved by the ROS in the microenvironment of cancer cells and/or hypertension, thereby release the two or more active agents in free form, without producing any byproduct.

The method for treating or ameliorating one or more symptoms associated with cancer and/or hypertension in a subject can follow the method step described above, for example, administering to the subject an effective amount of a pharmaceutical formulation containing one or more of the prodrugs by oral administration, parenteral administration, inhalation, mucosal, topical administration, or a combination thereof. In some forms, the method can include the additional step described above. For example, the user can administer one or more additional active agents, such as one or more anti-cancer or anti-hypertension agents, in their free form to the subject prior to, during, and/or subsequent to administering the pharmaceutical formulation to the subject. Anti-cancer and anti-hypertension agents are known, for example, see the National Cancer Institute database, “A to Z List of Cancer Drugs,” website cancer.gov/about-cancer/treatment/drugs and website healthline.com/health/high-blood-pressure-hypertension-medication.

The disclosed prodrugs, methods of using, and methods of making can be further understood through the following enumerated paragraphs.

1. A prodrug having the structure of:

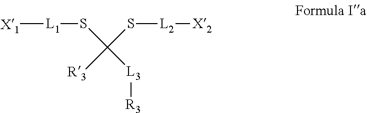

wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) X′₁ and X′₂ are independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl);
(c) R₃ is a substituted or unsubstituted aryl, a substituted or unsubstituted polyaryl, a substituted or unsubstituted heteroaryl, or a substituted or unsubstituted polyheteroaryl;
(d) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   2. The prodrug of paragraph 1, wherein X′₁-L₁- and X′₂-L₂- can be independently a moiety of Formula I″b:

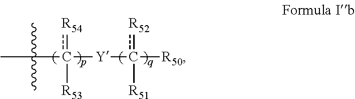

wherein:
(a) Y′ is a bond (single, double, or triple) or NR₅₅, and R₅₅ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(b) each occurrence of R₅₁ and R₅₃ is independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(c) each occurrence of R₅₂ and R₅₄ is independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(d) R₅₀ is hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl);
(e) p and q are independently an integer from 0 to 10, from 0 to 8, from 0 to 6, from 0 to 4, from 0 to 3, from 0 to 2, or 0 or 1; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   3. The prodrug of paragraph 2, wherein the moiety of Formula Ib has the structure of:

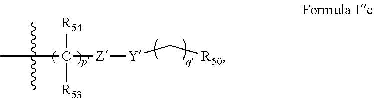

wherein:
(a) each occurrence of R₅₃ and R₅₄ is independently hydrogen, a substituted or unsubstituted alkyl, a substituted or unsubstituted aryl, a substituted or unsubstituted alkylaryl, a substituted or unsubstituted heterocyclyl, an amino, or a carbonyl;
(b) Z′ is a single bond or

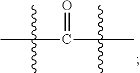 [SMILES: *C(*)=O]

(c) Y′ is a single bond or NR₅₅, and R₅₅ is hydrogen or a substituted or unsubstituted alkyl;
(d) R₅₀ is a carboxyl or a substituted or unsubstituted heterocyclyl (monoheterocyclyl or polyheterocyclyl);
(e) p′ and q′ are independently an integer from 0 to 3; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   4. The prodrug of paragraph 1, wherein the prodrug has the structure of:

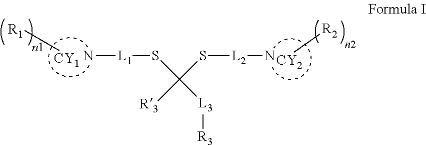

wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) CY₁ and CY₂ are independently a substituted or unsubstituted heteroaryl, a substituted or unsubstituted polyheteroaryl, or a substituted or unsubstituted heterocyclyl;
(c) R₁ and R₂ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether;
(d) n1 and n2 are independently an integer from 0 to 10;
(e) R₃ is a substituted or unsubstituted aryl, a substituted or unsubstituted polyaryl, a substituted or unsubstituted heteroaryl, or a substituted or unsubstituted polyheteroaryl;
(f) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   5. The prodrug of paragraph 4, wherein the prodrug has the structure of:

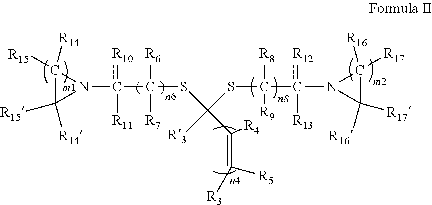

wherein:
(a) each occurrence of R₄-R₉, R₁₄-R₁₇, and R₁₄′-R₁₇′ is independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(b) m1 and m2 are independently an integer from 1 to 9;
(c) n4, n6, and n8 are independently an integer from 1 to 10;
(d) R₁₀ and R₁₂ are independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(e) R₁₁ and R₁₃ are independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(f) R′₃ is hydrogen, a substituted or unsubstituted alkyl, a substituted or unsubstituted aryl, a substituted or unsubstituted polyaryl, a substituted or unsubstituted heteroaryl, or a substituted or unsubstituted polyheteroaryl;
(g) R₃ is a substituted or unsubstituted aryl, a substituted or unsubstituted polyaryl, a substituted or unsubstituted heteroaryl, or a substituted or unsubstituted polyheteroaryl; and
(h) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   6. The prodrug of paragraph 4 or 5, wherein the prodrug has the structure of:

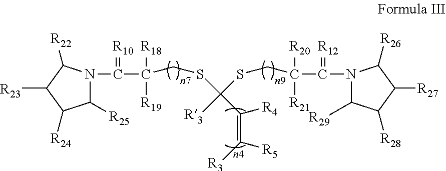

wherein:
(a) n7 and n9 are independently an integer from 0 to 8;
(b) R₁₈, R₁₉, R₂₀, and R₂₁ are independently hydrogen, a substituted or unsubstituted alkyl, a substituted or unsubstituted aryl, an alkoxyl, a hydroxyl, or a halogen;
(c) R₁₀ and R₁₂ are independently oxygen or sulfur;
(d) R₂₂-R₂₉ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol;
(e) n4 is an integer from 1 to 4;
(f) R₄ and R₅ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(g) R′₃ is hydrogen, a substituted or unsubstituted alkyl, a substituted or unsubstituted aryl, a substituted or unsubstituted polyaryl, a substituted or unsubstituted heteroaryl, or a substituted or unsubstituted polyheteroaryl;
(h) R₃ is a substituted or unsubstituted aryl, a substituted or unsubstituted polyaryl, a substituted or unsubstituted heteroaryl, or a substituted or unsubstituted polyheteroaryl; and
(i) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   7. The prodrug of paragraph 6, wherein at least one of R₁₈ and R₁₉ and/or at least one of R₂₀ and R₂₁ is a substituted or unsubstituted alkyl, a substituted or unsubstituted aryl, an alkoxyl, a hydroxyl, or a halogen.
   8. The prodrug of paragraph 6 or 7, wherein at least one of R₁₈ and R₁₉ and/or at least one of R₂₀ and R₂₁ is a substituted or unsubstituted alkyl.
   9. The prodrug of any one of paragraphs 6-8, wherein at least one of R₂₂ and R₂₅ and/or at least one of R₂₆ and R₂₈ is

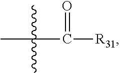

and R₃₁ is a hydroxyl, an alkoxyl, or an amino.
10. The prodrug of any one of paragraphs 5-9, wherein R₄ and R₅ are independently hydrogen, a substituted or unsubstituted alkyl, a substituted or unsubstituted aryl, an alkoxyl, a hydroxyl, or a halogen.
11. The prodrug of any one of paragraphs 1-10, wherein R₃ is

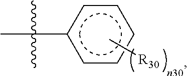

n30 is an integer from 0 to 5, and R₃₀ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether.
12. The prodrug of any one of paragraphs 4-11, wherein the prodrug has the structure of:

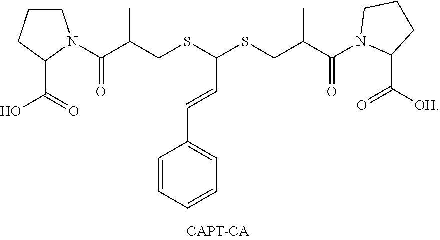 [SMILES: CC(CSC(/C=C/c1ccccc1)SCC(C)C(=O)N1CCCC1C(=O)O)C(=O)N1CCCC1C(=O)O]

13. A pharmaceutical formulation comprising
one or more prodrugs of any one of paragraphs 1-12; and
a pharmaceutically acceptable carrier and/or excipient,
wherein the one or more prodrugs are in an effective amount to prevent or treat an inflammatory disease or disorder, or treat or ameliorate one or more symptoms associated with an inflammatory disease or disorder in a subject.
   14. The pharmaceutical formulation of paragraph 13, wherein the pharmaceutically acceptable carrier is polymeric particles, and wherein the one or more prodrugs are encapsulated in, conjugated to, and/or complexed with the polymeric particles.
   15. The pharmaceutical formulation of paragraph 13 or 14 further comprising one or more additional active agents, and optionally wherein the one or more additional active agents is/are one or more anti-inflammatory agent.
   16. The pharmaceutical formulation of any one of paragraphs 13-15, wherein the total concentration of the one or more prodrugs in the pharmaceutical formulation is at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.
   17. A method of producing the prodrug of any one of paragraphs 1-12 comprising:
(i) heating a reaction mixture at a suitable temperature for a period of time sufficient to form a product comprising the prodrug,
wherein the reaction mixture comprises a first reactant, a second reactant, a third reactant, and a solvent, and wherein the first reactant is the same as or different from the second reactant.
   18. The method of paragraph 17, wherein the first reactant is X′₁-L₁-SH; the second reactant is X′₂-L₂-SH; and the third reactant has the structure of:

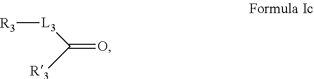

wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) X′₁ and X′₂ can be independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl);
(c) R₃ is a substituted or unsubstituted aryl, a substituted or unsubstituted polyaryl, a substituted or unsubstituted heteroaryl, or a substituted or unsubstituted polyheteroaryl;
(d) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   19. The method of paragraph 17 or 18, wherein the first reactant has the structure of:

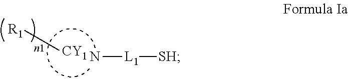

the second reactant has the structure of:

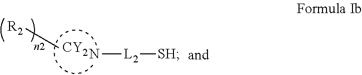

the third reactant has the structure of:

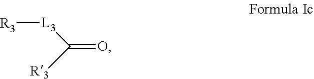

wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) CY₁ and CY₂ are independently a substituted or unsubstituted heteroaryl, a substituted or unsubstituted polyheteroaryl, or a substituted or unsubstituted heterocyclyl;
(c) R₁ and R₂ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether;
(d) n1 and n2 are independently an integer from 0 to 10;
(e) R₃ is a substituted or unsubstituted aryl, a substituted or unsubstituted polyaryl, a substituted or unsubstituted heteroaryl, or a substituted or unsubstituted polyheteroaryl;
(f) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   20. The method of any one of paragraphs 17-19, wherein the first reactant has the structure of:

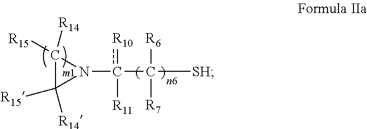

the second reactant has the structure of:

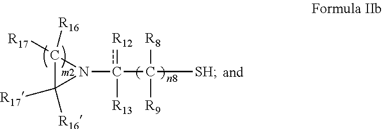

the third reactant has the structure of:

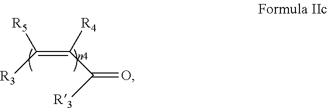

wherein:
(a) each occurrence of R₄-R₉, R₁₄-R₁₇, and R₁₄′-R₁₇′ is independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(b) m1 and m2 are independently an integer from 1 to 9;
(c) n4, n6, and n8 are independently an integer from 1 to 10;
(d) R₁₀ and R₁₂ are independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(e) R₁₁ and R₁₃ are independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(f) R′₃ is hydrogen, a substituted or unsubstituted alkyl, a substituted or unsubstituted aryl, a substituted or unsubstituted polyaryl, a substituted or unsubstituted heteroaryl, or a substituted or unsubstituted polyheteroaryl;
(g) R₃ is a substituted or unsubstituted aryl, a substituted or unsubstituted polyaryl, a substituted or unsubstituted heteroaryl, or a substituted or unsubstituted polyheteroaryl; and
(h) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   21. The method of paragraph 20, wherein the first reactant has the structure of:

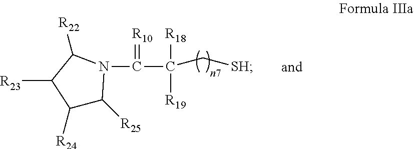

the second reactant has the structure of:

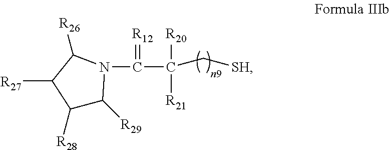

wherein:
(a) n7 and n9 are independently an integer from 0 to 8;
(b) R₁₈, R₁₉, R₂₀, and R₂₁ are independently hydrogen, a substituted or unsubstituted alkyl, a substituted or unsubstituted aryl, an alkoxyl, a hydroxyl, or a halogen;
(c) R₁₀ and R₁₂ are independently oxygen or sulfur;
(d) R₂₂-R₂₉ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.
   22. The method of paragraph 21, wherein at least one of R₁₈ and R₁₉ and/or at least one of R₂₀ and R₂₁ is a substituted or unsubstituted alkyl, a substituted or unsubstituted aryl, an alkoxyl, a hydroxyl, or a halogen.
   23. The method of 21 or 22, wherein at least one of R₁₈ and R₁₉ and/or at least one of R₂₀ and R₂₁ is a substituted or unsubstituted alkyl.
   24. The method of any one of paragraphs 21-23, wherein at least one of R₂₂ and R₂₅ and/or at least one of R₂₆ and R₂₈ is

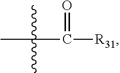

and R₃₁ is a hydroxyl, an alkoxyl, or an amino.
25. The method of any one of paragraphs 20-24, wherein R₄ and R₅ are independently hydrogen, a substituted or unsubstituted alkyl, a substituted or unsubstituted aryl, an alkoxyl, a hydroxyl, or a halogen.
26. The method of any one of paragraphs 18-25, wherein R₃ is

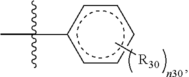

n30 is an integer from 0 to 5, and R₃₀ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether.
27. The method of any one of paragraphs 17-26, wherein each of the first reactant and the second reactant is captopril having the structure of:

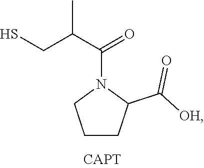

and
the third reactant is cinnamaldehyde having the structure of:

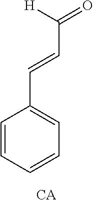

28. The method of any one of paragraphs 17-27, wherein the solvent is dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof.
29. The method of any one of paragraphs 17-28, wherein the reaction mixture further comprises a catalyst, and optionally wherein the catalyst is selected from the group consisting of catalyst is trifluoroacetic acid, p-toluene sulfonic acid, and boron trifluoride, and a combination thereof.
30. The method of any one of paragraphs 17-29, wherein the reaction mixture is cooled at a temperature in a range from 0° C. to −4° C., for a time period in a range from 12 hour to 36 hours.
31. The method of any one of paragraphs 17-30, further comprising mixing the first reactant, the second reactant, the third reactant, and the solvent to form the reaction mixture prior to step (i) and/or purifying the product containing the prodrug subsequent to step (i).
32. A method for preventing or treating an inflammatory disease or disorder, or treating or ameliorating one or more symptoms associated with an inflammatory disease or disorder in a subject in need thereof comprising
(i) administering to the subject the pharmaceutical formulation of any one of paragraphs 13-16,
wherein step (i) occurs one or more times.
   33. The method of paragraph 32 comprising only a single administration of the pharmaceutical formulation, wherein the pharmaceutical formulation comprises an effective amount of the prodrugs to change one or more clinical and/or biochemical measurements of the subject, compared to the subject before administered with the pharmaceutical formulation.
   34. The method of paragraph 32 comprising more than one step of administering to the subject the pharmaceutical formulation, wherein following all of the administration steps, an effective amount of the prodrugs to change one or more clinical and/or biochemical measurements of the subject, compared to the subject before administered with the pharmaceutical formulation, is administered to the subject.
   35. The method of any one of paragraphs 32-34, wherein the inflammatory disease or disorder is rheumatoid arthritis.
   36. A method for treating macrophages in a subject in need thereof comprising:
(i) administering to the subject the pharmaceutical formulation of any one of paragraphs 13-16,
wherein step (i) occurs one or more times.
   37. The method of paragraph 36 comprising only a single administration of the pharmaceutical formulation, wherein the pharmaceutical formulation comprises an effective amount of the prodrugs to reduce production of nitric oxide by macrophages and/or reduce release of one or more proinflammatory cytokines from the macrophages, compared with the production of nitric oxide by macrophages and/or release of the one or more proinflammatory cytokines from the macrophage in a control not treated with the prodrugs, tested under the same condition.
   38. The method of paragraph 36 comprising more than one step of administering to the subject the pharmaceutical formulation, wherein following all of the administration steps, an effective amount of the prodrugs to reduce production of nitric oxide by macrophages and/or reduce release of one or more proinflammatory cytokines from the macrophages is administered to the subject, compared with the production of nitric oxide by macrophages and/or release of the one or more proinflammatory cytokines from the macrophage in a control not treated with the prodrugs, tested under the same condition.
   39. A method for treating or ameliorating one or more symptoms associated with a cancer in a subject in need thereof comprising:
(i) administering to the subject the pharmaceutical formulation of any one of paragraphs 13-16,
wherein step (i) occurs one or more times.
   40. A method for treating or ameliorating one or more symptoms associated with hypertension in a subject in need thereof comprising:
(i) administering to the subject the pharmaceutical formulation of any one of paragraphs 13-16,
wherein step (i) occurs one or more times.
   41. The method of any one of paragraphs 32-40, wherein the subject is a mammal.
   42. The method of any one of paragraphs 32-41, wherein the pharmaceutical formulation is administered by oral administration, parenteral administration, inhalation, mucosal administration, topical or a combination thereof.
   43. The method of any one of paragraphs 32-42 further comprising administering to the subject one or more additional active agents prior to, during, and/or subsequent to step (i).

The present invention will be further understood by reference to the following non-limiting examples.

### EXAMPLES

### Example 1. Exemplary Prodrug CAPT-CA Shows Anti-Inflammatory Effects with Good Biocompatibility

### Materials and Methods

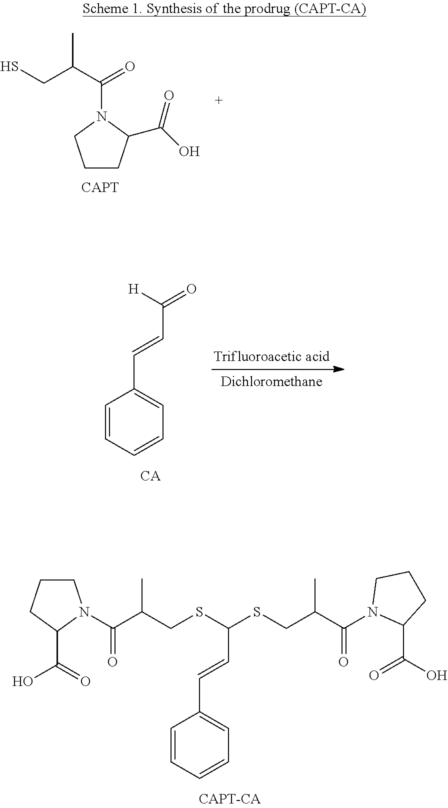 [SMILES: CC(CS)C(=O)N1CCCC1C(=O)O ; O=C/C=C/c1ccccc1 ; CC(CSC(/C=C/c1ccccc1)SCC(C)C(=O)N1CCCC1C(=O)O)C(=O)N1CCCC1C(=O)O]

### Synthesis

Cinnamaldehyde (0.6489 g, 4.91 mmol) and captopril (2.1956 g, 10.802 mmol) were dissolved in 20 mL of dichloromethane. 5 μL of trifluoroacetic acid was added under and ice bath condition. After the reaction at the ice bath in the dark for 24-48 h, the crude product was purified by column chromatography using ethyl acetate and hexane as eluents to give pure the prodrug product as a white solid.

### HPLC Measurements

HPLC spectra of CAPT, CA, CAPT-CA prodrug, and the degradation of CAPT-CA (160 μM) in the presence of H₂O₂ (40 mM) for different times were obtained. A C₁₈ column was used. The mobile phase A was acetonitrile containing 0.1% TFA and mobile phase B was water containing 0.1% TFA. The elution procedure was shown as below: 0-5 min: 98% B→98% B; 5-15 min: 98% B→40% B; 15-17 min: 40% B→40% B. The flow rate was 1.5 mL/min. The detection wavelength was 220 nm.

### Cytotoxicity Test

The effect of the prodrug on the viability of Raw 264.7 cells was determined using the MTT assay. Briefly, Raw 264.7 cells were seeded in 24-well plates. After a 24 h incubation, prodrugs were added to the cells at different concentrations and incubated for 1 h, and then, the cells were treated with or without LPS (10 μg/mL) for another 24 h. Next, 50 μL of MTT solution (5 mg/mL) were added to each well and the cells were incubated for 3 h at 37° C. Then, 1 mL of DMSO were added to dissolve the MTT-formazan crystal. The absorbance at 570 nm and 630 nm were measured. Three replicate wells were used in this experiment.

### Testing NO Generation

Nitrite levels, an index of cellular NO production, in culture supernatants of Raw 264.7 cells were determined using a modified Griess reagent. Briefly, Raw 264.7 cells were seeded in 24-well plates. After a 24 h incubation, prodrugs were added to the cells at different concentrations and incubated for 1 h, and then the cells were treated with or without LPS (10 μg/mL) for another 24 h. The supernatant was mixed with an equal volume of Griess reagent and incubated at room temperature for 15 min. The absorbance at 540 nm was measured. Three replicate wells were used in this experiment.

### Testing Protein Levels

The protein levels of TNF-α, IL-1β and IL-6 were determined by ELISA. Briefly, Raw 264.7 cells were seeded in 24-well plates. After a 24 h incubation, prodrugs were added to the cells at different concentrations and incubated for 1 h, and then the cells were treated with or without LPS (10 μg/mL) for another 24 h. The supernatants were collected for determination of protein levels according to the ELISA kit's instruction. Three replicate wells were used in this experiment.

Testing mRNA Levels

The mRNA levels of TNF-α, IL-1β and IL-10 were determined by qRT-PCR. Briefly, Raw 264.7 cells were seeded in 12-well plates. After a 24 h incubation, prodrugs were added to the cells at different concentrations and incubated for 1 h, and then the cells were treated with or without LPS (10 μg/mL) for another 24 h. qRT-PCR experiments were measured according to the manufacturer's instruction. Three replicate wells were used in this experiment.

### Results

The synthesis of the exemplary CAPT-CA prodrug is shown in Scheme 1. Proton nuclear magnetic resonance (¹H NMR) spectrum (not shown) has confirmed the successful synthesis of the prodrug. ¹H NMR (400 MHz, d6-DMSO) δ (ppm): 7.37-7.24 (m, 5H), 6.60 (dd, J=6 Hz, 12 Hz, 1H), 6.21 (dd, J=6 Hz, 12 Hz, 1H), 4.76 (d, J=9 Hz, 1H), 4.26-4.16 (m, 2H), 3.56-3.51 (m, 6H), 1.91-1.77 (m, 8H), 1.13-0.97 (m, 6H).

To evaluate the ROS-responsiveness of CAPT-CA, high performance liquid chromatography (HPLC) has been employed to monitor the release of CAPT and CA after incubation of CAPT-CA with a ROS reagent (hydrogen peroxide, H₂O₂). The retention time of free CAPT, free CA and CAPT-CA was at 10.138 min, 13.275 min, 14.283 min, respectively. After incubation with H₂O₂ for 4 h, free CA and CAPT peaks are shown while the prodrug peak decreases, demonstrating that some thioacetals have been cleaved by H₂O₂. The prodrug peak almost disappeared after 22 h, demonstrating that the prodrug shows a time-dependent ROS-responsiveness and can be active under ROS condition to release free drugs.

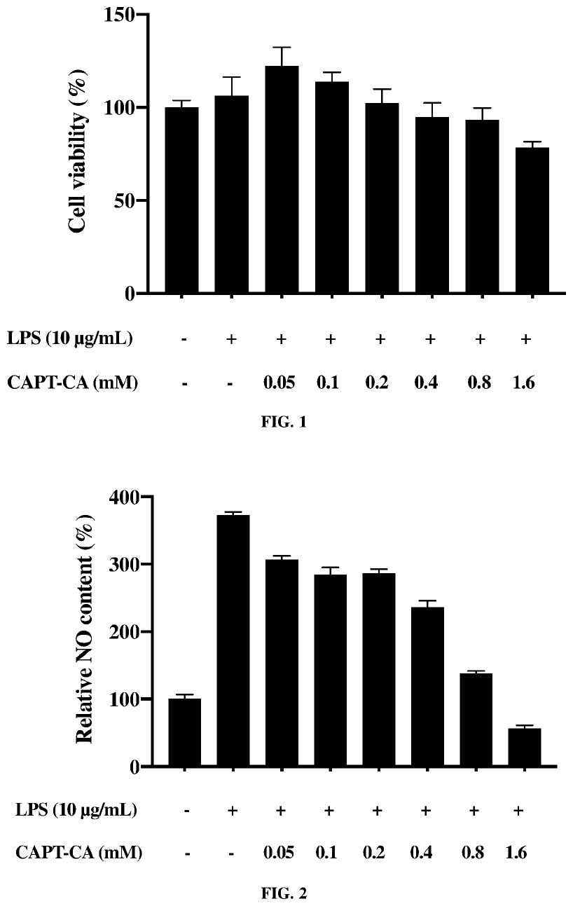

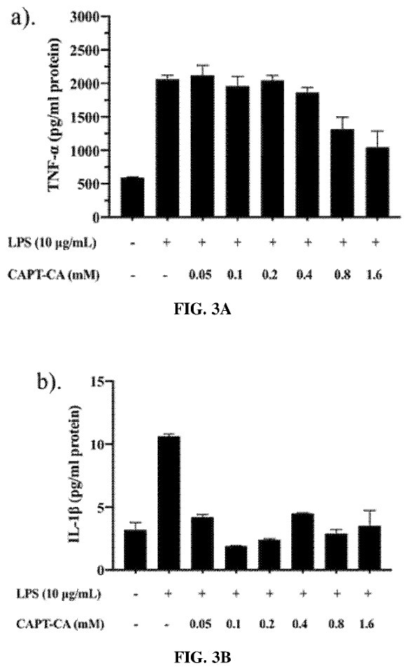

The biocompatibility and therapeutic efficacy of the CAPT-CA prodrug in lipopolysaccharide (“LPS”)-induced macrophages have been evaluated. The cytotoxicity of the prodrug is minimal, which demonstrates the good biocompatibility of the prodrug in the tested concentration range (FIG. 1). Nitric oxide (“NO”) is an indicator of inflammatory responses. Measuring the production of LPS-induced NO can be used for evaluating the progression of inflammation; inhibition of NO production has potential therapeutic value for controlling inflammatory reactions and diseases. As shown in FIG. 2, the increase in NO generation resulting from LPS stimulation in Raw264.7 cells is significantly suppressed by CAPT-CA treatment in a dose-dependent manner.

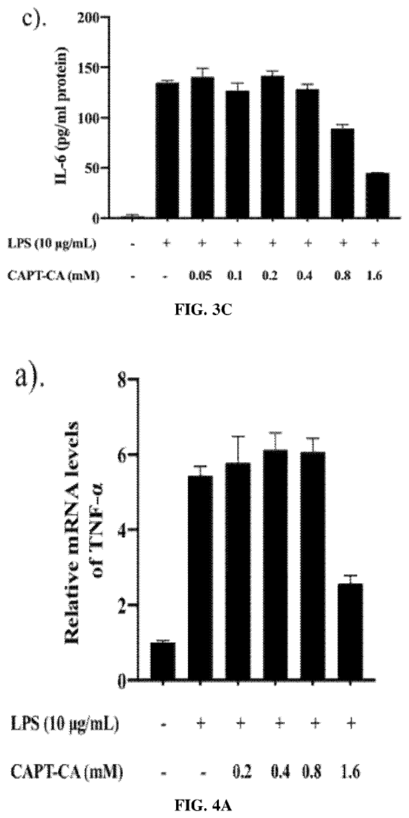

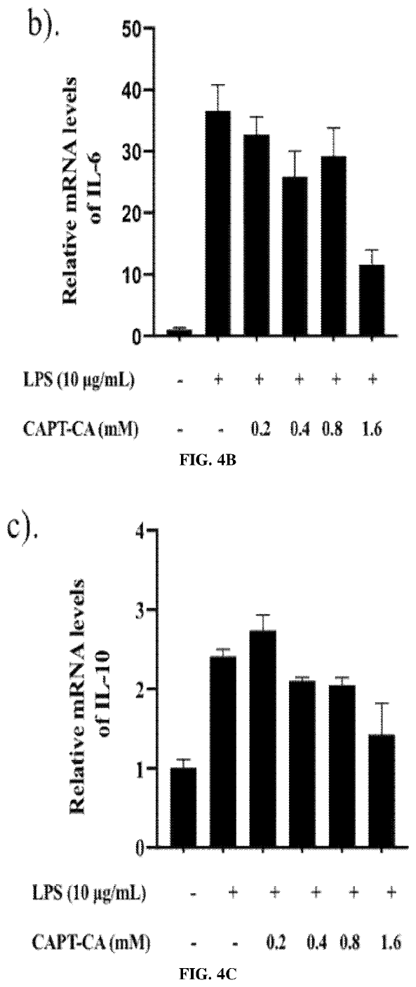

Further, LPS-activated Raw264.7 cells release proinflammatory cytokines, such as IL-1β, IL-6, and TNF-α, which play important roles in the pathogenesis of inflammatory conditions. Enzyme-linked immunosorbent assay (ELISA) and real-time quantitative polymerase chain reaction (RT-qPCR) have been performed to measure the protein and mRNA levels of these inflammatory cytokines, respectively. FIGS. 3A-3C and 4A-4C show that the CAPT-CA prodrug significantly down-regulates the protein and mRNA levels of the cytokines in LPS-stimulated Raw264.7 cells in a dose dependent manner. These results demonstrate that CAPT-CA has anti-inflammatory effects with good biocompatibility.

The CAPT-CA prodrug is the first ROS-sensitive prodrug that can be cleaved by oxidative stress to release two free drugs without producing any by-products. The CAPT-CA prodrug can be used in the treatment of rheumatoid arthritis. The CAPT-CA prodrug can also be used in the treatment of other diseases, such as cancer and hypertension, since both CAPT and CA have other therapeutic uses.

### REFERENCES

Syed, et al. Journal of Drug Delivery Science and Technology 2019, 53.
Tarner and Müller-Ladner, Expert opinion on drug delivery 2008, 5 (9), 1027-1037.
Mohsenzadegan, Iran J Allergy Asthma Immunol 2008, 7 (4), 195-202.
da Fonseca, et al. Oxid Med Cell Longev 2019, 2019, 7536805.
Andersen, et al. Eur J Med Chem 2018, 156, 738-746.
Previtali, et al. Bioorg Med Chem 2020, 28 (2), 115247.
Peiro Cadahia, et al. J Med Chem 2018, 61 (8), 3503-3515.
Martin, et al. The lancet 1984.
Ankur Gupta, et al. Tropical Journal of Pharmaceutical Research 2007, 6 (2), 687-693.
Xu, et al. Biomacromolecules 2018, 19 (12), 4658-4667.
Li and Wang, Inflammation 2020, 43 (5), 1729-1741.
Cheng, et al. J Pharmacol Exp Ther 2020, 373 (2), 302-310.
Mateen, et al. Phytomedicine 2019, 53, 70-78.
Noh, et al. Nat Commun 2015, 6, 6907.

1. A prodrug having the structure of: 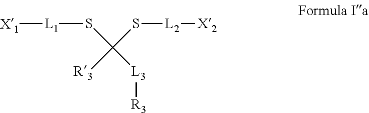
wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) X′₁ and X′₂ are independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl);
(c) R₃ is a substituted or unsubstituted aryl, a substituted or unsubstituted polyaryl, a substituted or unsubstituted heteroaryl, or a substituted or unsubstituted polyheteroaryl;
(d) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

2. The prodrug of claim 1, wherein X′₁-L₁- and X′₂-L₂- can be independently a moiety of Formula I″b: 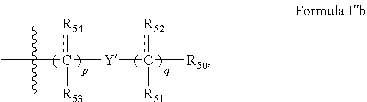
wherein:
(a) Y′ is a bond (single, double, or triple) or NR₅₅, and R₅₅ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(b) each occurrence of R₅₁ and R₅₃ is independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(c) each occurrence of R₅₂ and R₅₄ is independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(d) R₅₀ is hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl);
(e) p and q are independently an integer from 0 to 10, from 0 to 8, from 0 to 6, from 0 to 4, from 0 to 3, from 0 to 2, or 0 or 1; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

3. The prodrug of claim 2, wherein the moiety of Formula I″b has the structure of: 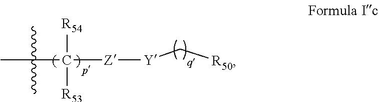
wherein:
(a) each occurrence of R₅₃ and R₅₄ is independently hydrogen, a substituted or unsubstituted alkyl, a substituted or unsubstituted aryl, a substituted or unsubstituted alkylaryl, a substituted or unsubstituted heterocyclyl, an amino, or a carbonyl;
(b) Z′ is a single bond or 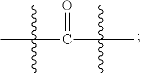 [SMILES: *C(*)=O]
(c) Y′ is a single bond or NR₅₅, and R₅₅ is hydrogen or a substituted or unsubstituted alkyl;
(d) R₅₀ is a carboxyl or a substituted or unsubstituted heterocyclyl (monoheterocyclyl or polyheterocyclyl);
(e) p′ and q′ are independently an integer from 0 to 3; and
(f) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

4. The prodrug of claim 1, wherein the prodrug has the structure of: 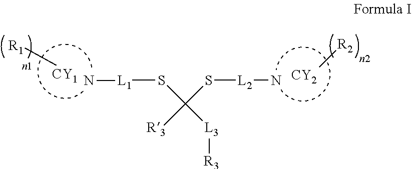
wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) CY₁ and CY₂ are independently a substituted or unsubstituted heteroaryl, a substituted or unsubstituted polyheteroaryl, or a substituted or unsubstituted heterocyclyl;
(c) R₁ and R₂ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether;
(d) n1 and n2 are independently an integer from 0 to 10;
(e) R₃ is a substituted or unsubstituted aryl, a substituted or unsubstituted polyaryl, a substituted or unsubstituted heteroaryl, or a substituted or unsubstituted polyheteroaryl;
(f) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(g) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

5. The prodrug of claim 4, wherein the prodrug has the structure of: 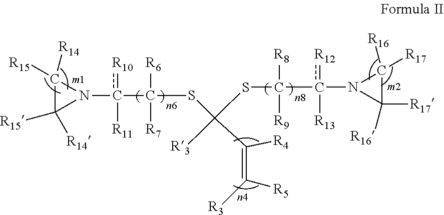
wherein:
(a) each occurrence of R₄-R₉, R₁₄-R₁₇, and R₁₄′-R₁₇′ is independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(b) m1 and m2 are independently an integer from 1 to 9;
(c) n4, n6, and n8 are independently an integer from 1 to 10;
(d) R₁₀ and R₁₂ are independently oxygen, sulfur, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(e) R₁₁ and R₁₃ are independently absent, hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(f) R′₃ is hydrogen, a substituted or unsubstituted alkyl, a substituted or unsubstituted aryl, a substituted or unsubstituted polyaryl, a substituted or unsubstituted heteroaryl, or a substituted or unsubstituted polyheteroaryl;
(g) R₃ is a substituted or unsubstituted aryl, a substituted or unsubstituted polyaryl, a substituted or unsubstituted heteroaryl, or a substituted or unsubstituted polyheteroaryl; and
(h) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

6. The prodrug of claim 4, wherein the prodrug has the structure of: 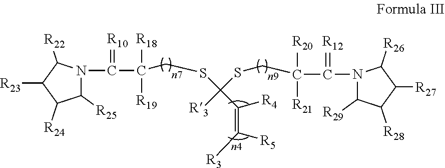
wherein:
(a) n7 and n9 are independently an integer from 0 to 8;
(b) R₁₈, R₁₉, R₂₀, and R₂₁ are independently hydrogen, a substituted or unsubstituted alkyl, a substituted or unsubstituted aryl, an alkoxyl, a hydroxyl, or a halogen;
(c) R₁₀ and R₁₂ are independently oxygen or sulfur;
(d) R₂₂-R₂₉ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an alkoxy, a carbonyl, a nitro, or a thiol;
(e) n4 is an integer from 1 to 4;
(f) R₄ and R₅ are independently hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, or a thiol;
(g) R′₃ is hydrogen, a substituted or unsubstituted alkyl, a substituted or unsubstituted aryl, a substituted or unsubstituted polyaryl, a substituted or unsubstituted heteroaryl, or a substituted or unsubstituted polyheteroaryl;
(h) R₃ is a substituted or unsubstituted aryl, a substituted or unsubstituted polyaryl, a substituted or unsubstituted heteroaryl, or a substituted or unsubstituted polyheteroaryl; and
(i) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

7. The prodrug of claim 6, wherein at least one of R₁₈ and R₁₉ and/or at least one of R₂₀ and R₂₁ is a substituted or unsubstituted alkyl, a substituted or unsubstituted aryl, an alkoxyl, a hydroxyl, or a halogen.

8. (canceled)

9. The prodrug of claim 6, wherein at least one of R₂₂ and R₂₅ and/or at least one of R₂₆ and R₂₈ is 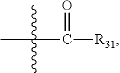
and R₃₁ is a hydroxyl, an alkoxyl, or an amino.

10. The prodrug of claim 5, wherein R₄ and R₅ are independently hydrogen, a substituted or unsubstituted alkyl, a substituted or unsubstituted aryl, an alkoxyl, a hydroxyl, or a halogen.

11. The prodrug of claim 1, wherein R₃ is 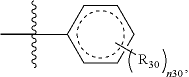
n30 is an integer from 0 to 5, and R₃₀ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, a halogen, a hydroxyl, an oxo, an amino, an azido, an alkoxy, a cyano, an isocyano, a carbonyl, a nitro, a thiol, a polyether, or a thioether.

12. The prodrug of claim 4, wherein the prodrug has the structure of:

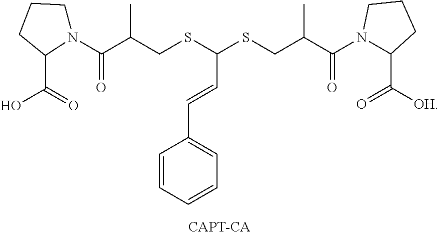 [SMILES: CC(CSC(/C=C/c1ccccc1)SCC(C)C(=O)N1CCCC1C(=O)O)C(=O)N1CCCC1C(=O)O]

13. A pharmaceutical formulation comprising
one or more prodrugs of claim 1; and
a pharmaceutically acceptable carrier and/or excipient,
wherein the one or more prodrugs are in an effective amount to prevent or treat an inflammatory disease or disorder, or treat or ameliorate one or more symptoms associated with an inflammatory disease or disorder in a subject.

14.-16. (canceled)

17. A method of producing the prodrug of claim 1, comprising:
(i) heating a reaction mixture at a suitable temperature for a period of time sufficient to form a product comprising the prodrug,
wherein the reaction mixture comprises a first reactant, a second reactant, a third reactant, and a solvent, and wherein the first reactant is the same as or different from the second reactant,
wherein the first reactant is X′₁-L₁-SH; the second reactant is X′₂-L₂-SH; and the third reactant has the structure of: 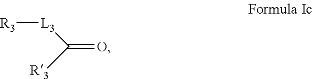
wherein:
(a) L₁, L₂, and L₃ are independently a bond (single, double, or triple), a substituted or unsubstituted alkyl, a substituted or unsubstituted alkenyl, an amino, an amido, a carbonyl, an alkoxyl, a polyether, a thioether;
(b) X′₁ and X′₂ can be independently hydrogen, a carboxyl, a substituted or unsubstituted heteroaryl, a substituted or unsubstituted polyheteroaryl, or a substituted or unsubstituted heterocyclyl (monoheterocyclyl or polyheterocyclyl):
(c) R₃ is a substituted or unsubstituted aryl, a substituted or unsubstituted polyaryl, a substituted or unsubstituted heteroaryl, or a substituted or unsubstituted polyheteroaryl:
(d) R′₃ is hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted polyheteroaryl, a substituted or unsubstituted heterocyclyl, an amino, a hydroxyl, or an alkoxy; and
(e) the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, or a thiol.

18.-27. (canceled)

28. The method of claim 17, wherein the solvent is dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, tetrahydrofuran, 2-methyl tetrahydrofuran, acetonitrile, or toluene, or a combination thereof.

29. The method of claim 17, wherein the reaction mixture further comprises a catalyst, and optionally wherein the catalyst is selected from the group consisting of catalyst is trifluoroacetic acid, p-toluene sulfonic acid, and boron trifluoride, and a combination thereof.

30. The method of claim 17, wherein the reaction mixture is cooled at a temperature in a range from 0° C. to −4° C., for a time period in a range from 12 hour to 36 hours.

31. (canceled)

32. A method for preventing or treating an inflammatory disease or disorder, or treating or ameliorating one or more symptoms associated with an inflammatory disease or disorder in a subject in need thereof comprising:
(i) administering to the subject the pharmaceutical formulation of claim 13, wherein step (i) occurs one or more times.

33.-35. (canceled)

36. A method for treating macrophages in a subject in need thereof comprising:
(i) administering to the subject the pharmaceutical formulation of claim 13, wherein step (i) occurs one or more times.

37.-38. (canceled)

39. A method for treating or ameliorating one or more symptoms associated with a cancer in a subject in need thereof comprising:
(i) administering to the subject the pharmaceutical formulation of claim 13, wherein step (i) occurs one or more times.

40. A method for treating or ameliorating one or more symptoms associated with hypertension in a subject in need thereof comprising:
(i) administering to the subject the pharmaceutical formulation of claim 13, wherein step (i) occurs one or more times.

41.-43. (canceled)
